(19) United States
(12) Patent Application Publication
(10) Pub. No.: US 2024/0267494 A1
(43) Pub. Date: Aug. 8, 2024

(54) INTRAOCULAR SYSTEM THAT INCLUDES AN IMPLANT WITH AN IMAGE FORMATION DEVICE

(71) Applicant: Twenty Twenty Therapeutics LLC, South San Francisco, CA (US)

(72) Inventors: Supriyo SINHA, Menlo Park, CA (US); Kirk Gossage, Pacifica, CA (US); Shungneng Lee, South San Francisco, CA (US); Dimitri Azar, Chicago, IL (US)

(21) Appl. No.: 18/164,484

(22) Filed: Feb. 3, 2023

(51) Int. Cl. H04N 7/18; A61F 2/14; G06F 3/01; G06V 20/40; G06V 20/50; H04N 5/262; H04N 23/90

(52) U.S. Cl. CPC H04N 7/183; A61F 2/141; A61F 2/1602; A61F 9/08; G06F 3/013; G06F 3/017; G06V 20/40; G06V 20/50; H04N 5/2628; H04N 23/90; A61F 2250/0002; G02B 27/0172

(57) ABSTRACT

A method performed by a headset that is part of an intraocular system that includes an implant that includes an image formation device. The headset captures, by a camera of the headset that is being worn by a user, a first video stream that includes a visual representation of an environment of the headset. The headset determines whether the first video stream is to be enhanced, and responsive to determining that the first video stream is to be enhanced, producing a second video stream that includes an enhanced visual representation of the environment. The headset transmits, via a wireless connection, the second video stream to the implant that is inside an eye of the user, wherein the implant is configured to use the image formation device to present the second video stream.

### FIELD

An embodiment of the disclosure relates to an intraocular system that includes an implant with an image formation device for presenting video (e.g., a reproduction of one or more optical images) on a retina of a person who may be vision impaired. Other embodiments are also described.

### BACKGROUND

Disease or injury to a cornea of a person can lead to opacification or significant optical damage to the cornea, such that the individual may be vision impaired (e.g., effectively rendered blind). Current solutions rely on eye banks for corneal transplants, and artificial corneas. Both however have issues with transplant rejection and surgical complications, and may also result in low visual acuity.

Blindness due to corneal disease or injury may occur despite the person having a fully functioning retina. For such persons, who have a functioning retina but otherwise are essentially blind due to vascularization or damage to the cornea, implantation of an intraocular projector into may restore vision to the person. The intraocular projector receives an image of a scene before the person, the image having been captured by a head mounted camera, and then projects the image onto the retina of the eye. Another option is an implantation of an intraocular micro-display into the lens of the eye (e.g., into the capsular bag region), which may receive and display the image onto the user's retina.

### SUMMARY

One embodiment of the disclosure is an intraocular system that includes a headset and an implant that may provide a (visually impaired, due to having opaque corneas, for example) user a visual reproduction of a scene of an environment in which the user is located. In particular, the headset may include a camera that captures video (images) of a scene that is before the person. For example, the camera may have a field of view that is directed away from and in front of the user, while the headset is worn. The headset may wirelessly transmit (e.g., over the air) the video to the implant, which may then use an image formation device, such as a micro-display, to present the video towards the user's retina.

An intraocular system may be capable of providing a visually impaired user with a better or improved visual acuity by presenting color images onto the user's retina. In which case, the user may view a scene in front of the user in a similar (or same) fashion as a person with “normal” vision (e.g., without the visual impairment and/or without the use of the intraocular system). In some situations, it may be desirable to view an augmented or enhanced scene, as opposed to only viewing a scene that would be similarly perceived (e.g., as color images) by a person with normal vision. Therefore, there is a need for augmenting or enhancing a user's visual experience by showing different and/or additional information through the use of the image formation device of an intraocular implant.

The present disclosure provides a method and an example intraocular system for determining whether to augment or enhance video (images) captured by a camera of a headset, for presentation (display) by an implant inside a user's eye. In particular, the headset captures, by a camera, a first video stream that includes a visual representation of an outside environment of the headset, and determines whether the first video stream is to be augmented or enhanced. For example, in response to detecting an object that is within a close vicinity of the user (e.g., within a threshold distance), the first video stream may be augmented by adding (e.g., superimposing) a notification on the video to notify the user of the object. As another example, the headset may enhance the first video stream by performing one or more video processing operations, such applying a brightness enhancement when the captured video is dark (e.g., having a brightness below a threshold). As a result, the headset may produce a second video stream that includes an augmented or enhanced version of the first video stream, responsive to determining that the first video stream is to be augmented or enhanced. The headset may transmit, via a wireless connection (e.g., BLUETOOTH), the second video stream to the implant of the intraocular system for presentation by an image formation device (e.g., micro-display) of the implant. Thus, the present disclosure provides a user who may otherwise be visually impaired, an augmented or enhanced visual experience through the use of an intraocular implant.

In one embodiment, the headset receives, from a proximity sensor of the headset, proximity data that indicates a distance from an object within the environment, where determining whether the first video stream is to be enhanced includes determining that the distance is within a threshold distance. In another embodiment, the enhanced visual representation of the environment comprises a notification to notify the user of a presence of the object within the environment. In some embodiments, determining whether the video is to be enhanced includes determining that one or more characteristics of the first video stream exceed a threshold. In one embodiment, producing the second video stream includes performing one or more video processing operations upon the first video stream such that the one or more characteristics remain below the threshold. In another embodiment, the one or more video processing operations comprises at least one of a brightness enhancement, a contrast enhancement, and a color enhancement.

In one embodiment, the camera is a first camera, where the second video stream is produced by a second camera that is part of the headset. In another embodiment, the second video stream includes a thermal image of a portion of the environment when the second camera is a thermal imaging camera. In some embodiments, the second video stream includes a depth map of a portion of the environment with respect to the headset when the second camera is a depth camera. In another embodiment, a determination that the video is to be enhanced is responsive to receiving user input. In some embodiments, determining whether the first video stream is to be enhanced includes detecting, using an object recognition algorithm, an object within the environment, wherein producing the second video stream comprises adding a notification relating to the object to a portion of the first video stream. In one embodiment, adding the notification comprises highlighting a region surrounding the object within the video stream to emphasis the object. In another embodiment, the image formation device comprises a micro-display with a plurality of pixel arrays or is a laser projector.

In one embodiment, one of the challenges with an intraocular system is optimizing power consumption of the headset and of the intraocular implant. For example, the headset may include a power source (e.g., an on-board battery), with which the headset uses to power on-board electronics as well as supplying power to the implant (e.g., through wireless power transfer). To conserve power by reducing power consumption of the implant, it may be desirable to not always have the implant on or active. For instance, it may be desirable to deactivate the image formation device of the implant under certain conditions (e.g., while the user naps). Therefore, there is a need for an intraocular system that is capable of determining whether to deactivate (at least a portion) of the system.

The present disclosure provides a method and an example intraocular system for determining whether to deactivate (or reduce power consumption of) an intraocular implant. Specifically, the headset captures a video stream and transmits the video stream to the implant for presentation by the image formation device (e.g., micro-display). The headset detects a gesture performed by the user. For instance, the gesture may be a series of blinks, or may be user input through an input device (e.g., a button) of the headset. The headset determines whether the image formation device is to be deactivated or remain activate based on the detected gesture. Returning to the previous example, the headset may determine whether the series of blinks is associated with a user command to shut off (or down) the implant, or if the blinks are a natural series of blinks. If determined that the image formation device is to be deactivated (e.g., based on the detected gesture), the headset may transmit a control signal to the implant to cause the device to deactivate. As a result, the intraocular system may reduce power consumption while the image device is deactivated. In addition, such a system may provide the user with an ability to manually (and automatically) deactivate the implant. For instance, a user may desire to deactivate the implant while traveling in a dimly lit room. This may also allow the user to deactivate the implant, such that the user has little or no visual acuity, while keeping the user's eyes open.

In one embodiment, the camera is a first camera that has a first field-of-view (FOV) directed away from the user when the headset is worn by the user, where the headset includes a second camera that has a second FOV directed towards the eye of the user when the headset is worn by the user, where detecting the gesture includes performing a gesture detection algorithm to detect an eye gesture of the eye of the user. In another embodiment, the image formation device is determined to be deactivated when the eye gesture is a gaze for a period of time. In some embodiments, the image formation device is determined to be deactivated when the eye gesture is a series of blinks or winks. In another embodiment, the second FOV is directed towards both eyes of the user, where the eye gesture comprises both eyes closing, where the image formation device is determined to be deactivated responsive to determining that both eyes are closed and remain closed for a period of time. In some embodiments, the gestures is a first gesture, where the headset further detects a second gesture performed by the user, where the image formation device is determined to remain active while both eyes are closed responsive to the detecting of the second gesture.

In one embodiment, the eye is a first eye and the gesture is a first gesture, where the control signal is a first control signal, where the gesture comprises the second eye of the user closing, where the headset further: detects an eye-closure condition within the environment; determines whether the user has performed a second gesture; responsive to determining that the user has performed the second gesture, the detecting of the eye-closure condition, and that the user's second eye is closed, determines that the image formation device is to be deactivated. In another embodiment, determining that the image formation device is to be deactivated includes ceasing to transmit the video stream to the implant, where the headset further determining that the image formation device is to remain active responsive to determining that the user has not performed the second gesture, the detecting of the eye-closure condition, and that the user's second eye is closed; and responsive to determining that the image formation device is to remain active, continuing to transmit the video stream to the implant for presentation by the image formation device. In one embodiment, the eye-closure condition comprises at least one of an environmental condition that causes low visibility, and a lighting condition with a brightness that exceeds a threshold. In some embodiments, the control signal causes the image formation device to be deactivated by instructing the image formation device to cease displaying the video stream. In another embodiment, the headset further transmits, via the wireless connection, an image to the implant to cause the image formation device to present the image.

As described herein, the image formation device of the implant may be a micro-display that includes a configuration of a two-dimensional (2D) array of pixel cells (e.g., light emitting diodes (LED)). In one embodiment, the pixel count of the micro-display may be smaller than the visible field (e.g., which may include a foveal vision area) of the human eye. As a result, some objects or features that are reproduced through the micro-display may have low (or lower than desired) visual acuity. Therefore, there is a need for zooming into captured video to provide the user with higher acuity (while at the expense of decreasing the angular visual field). The present disclosure provides a method and an example intraocular system that is capable of zooming into portions of video to provide the higher acuity, and that is capable of zooming out so that the entire visual field may be presented onto the user's retina. In particular, the headset of the system captures the first video stream and transmits the video stream to the implant to cause the implant's image formation device to present the video stream. The headset determines a region of interest within the first video stream, such as a portion surrounding a center of a video frame. The headset produces a second video stream by zooming into the region of interest (e.g., performing a zooming operation in order to focus into the region of interest. The headset transmits the second video stream to the implant for presentation.

In one embodiment, the headset further determines that the region of interest is to be zoomed out; produces a third video stream by zooming out of the region of interest; and transmits, via the wireless connection, the third video stream in lieu of the second video stream to the implant for display on the image formation device. In another embodiment, the third video stream is the first video stream. In some embodiments, the headset receives, from a sensor of the headset, sensor data based on the environment, where the region of interest is determined to be zoomed out based on the sensor data. In another embodiment, the sensor is an accelerometer and the sensor data is motion data, where determining that the region of interest is to be zoomed out includes determining that the user is in motion based on the motion data. In another embodiment, the sensor is a proximity sensor and the sensor data is proximity data, where determining that the region of interest is to be zoomed out includes determining that an object is within a threshold distance of the headset based on the proximity data. In some embodiments, the image formation device comprises a configuration of a two-dimensional (2D) array of a plurality of pixel cells. In one embodiment, the image formation device comprises a scanning projection system.

The above summary does not include an exhaustive list of all embodiments of the disclosure. It is contemplated that the disclosure includes all systems and methods that can be practiced from all suitable combinations of the various embodiments summarized above, as well as those disclosed in the Detailed Description below and particularly pointed out in the claims. Such combinations may have particular advantages not specifically recited in the above summary.

### BRIEF DESCRIPTION OF THE DRAWINGS

The embodiments are illustrated by way of example and not by way of limitation in the figures of the accompanying drawings in which like references indicate similar elements. It should be noted that references to “an” or “one” embodiment of this disclosure are not necessarily to the same embodiment, and they mean at least one. Also, in the interest of conciseness and reducing the total number of figures, a given figure may be used to illustrate the features of more than one embodiment, and not all elements in the figure may be required for a given embodiment.

FIG. 1 is a plan view illustration of a user and an example intraocular system that includes a headset and an implant.

FIG. 2 is a side-view illustration of the user and the example intraocular system.

FIG. 3 is a cross-sectional illustration of the user and the example intraocular system in which the implant is presenting video.

FIG. 4 shows a block diagram of the example intraocular system.

FIG. 5 is a signal diagram of one embodiment of a process by the headset and implant of the example intraocular system to augment or enhance captured video for wireless transmission to the implant.

FIG. 6 is a flowchart of one embodiment of a process to determine whether to augment or enhance the captured video for presentation by the implant

FIG. 7 is a signal diagram of one embodiment of a process by the headset and the implant of the example intraocular system to deactivate the image formation device of the implant.

FIG. 8 is a flowchart of one embodiment of a process to determine whether to deactivate the image formation device of the implant.

FIG. 9 is a flowchart of another embodiment of a process to determine whether to deactivate the image formation device of the implant.

FIG. 10 is a signal diagram of one embodiment of a process by the headset and the implant of the example intraocular system to perform a zoom operation.

FIG. 11 is a flowchart of one embodiment of a process to determine whether to zoom into the video stream captured by the camera.

FIG. 12 is a flowchart of one embodiment of a process to determine whether to perform a zoom-out operation.

FIG. 13 is a flowchart of one embodiment of a process performed by the implant to determine process a received video stream based on sensor data.

### DETAILED DESCRIPTION

Several embodiments of the disclosure with reference to the appended drawings are now explained. Whenever the shapes, relative positions and other embodiments of the parts described in a given embodiment are not explicitly defined, the scope of the disclosure here is not limited only to the parts shown, which are meant merely for the purpose of illustration. Also, while numerous details are set forth, it is understood that some embodiments may be practiced without these details. In other instances, well-known circuits, structures, and techniques have not been shown in detail so as not to obscure the understanding of this description. Furthermore, unless the meaning is clearly to the contrary, all ranges set forth herein are deemed to be inclusive of each range's endpoints.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Embodiments of the intraocular system disclosed herein may be suitable for patients with intact retinas, yet are blind due to vascularization, occlusion, opacity, or otherwise damage of the cornea. The disclosed system seeks to (at least partially) restore sight to these patients by implanting an electronic intraocular implant (which may be referred to herein as “implant”) into the eye, such as in the capsular sack region of the eye previously occupied by an excised lens. As described herein, the implant may include an image formation device, such as a micro-display that is arranged to project regenerated images onto the patient's (e.g., fully functioning) retina.

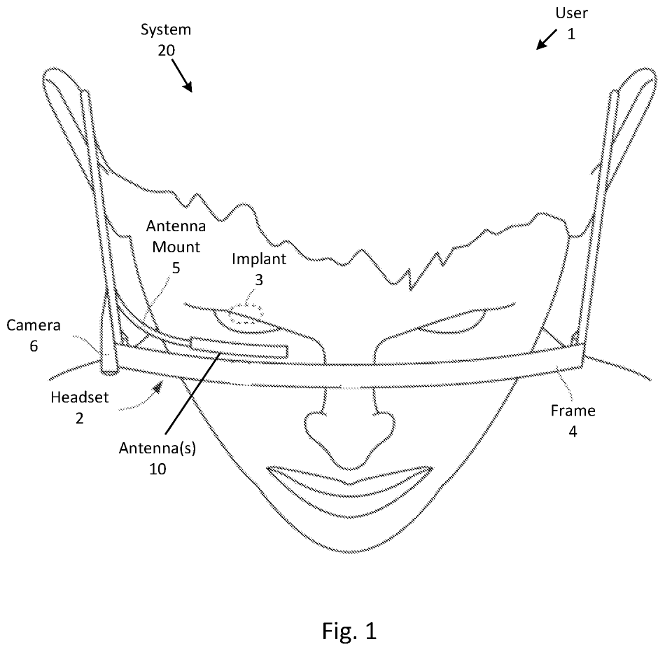

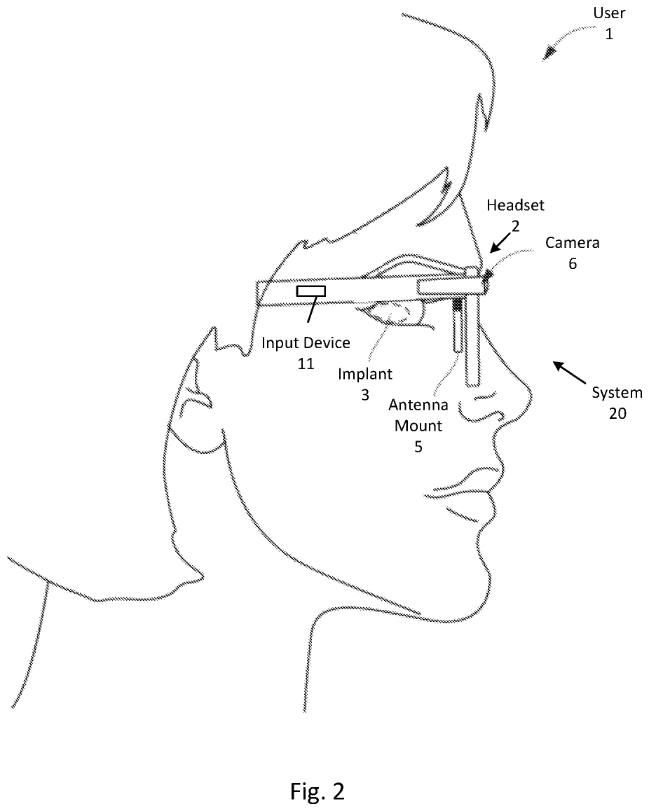

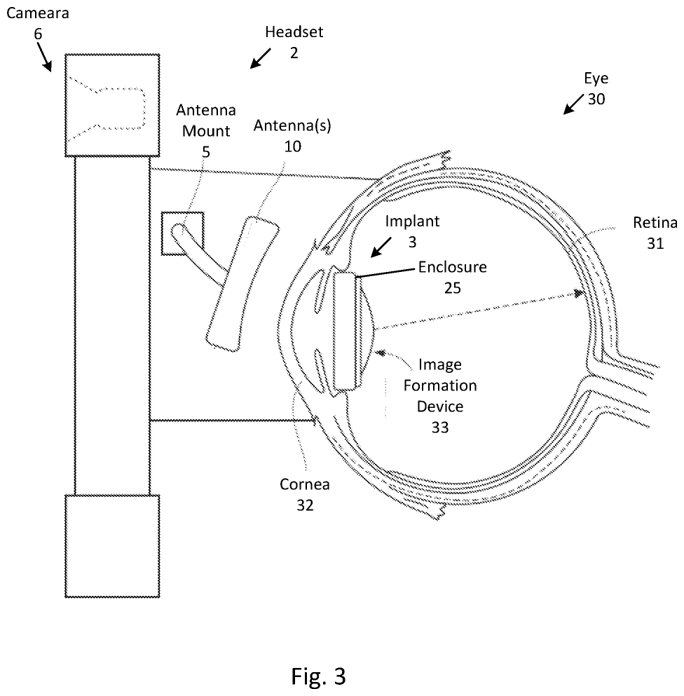

FIGS. 1-3 show a user 1 and an example intraocular system (hereafter may be referred to as “system”) 20 that includes a headset 2 and an implant 3, in accordance with an embodiment of the disclosure. FIGS. 1 and 2 are plan and side view illustrations, respectively, while FIG. 3 is a cross-sectional illustration of the system 20. As described herein, the intraocular system 20 may be configured to capture, using one or more cameras of the headset, video data (e.g., a reproduction of one or more optical images) that includes a visual representation of the environment (e.g., which may be in a frontal or forward direction of the user), and may be configured to optically present (or display) the captured video data through the implant 3 onto the retina of the user. More about presenting video data onto the retina of the user is described herein.

As shown, the illustrated headset 2 is eye glasses that are being worn by (e.g., on the head of) the user 1, and the implant 3 is (implanted or at least partially) inside the (e.g., capsular sack region of the) user's right eye. In one embodiment, the user 1 may include one or more implants, such as having one implant in the user's right eye (as shown), and another implant in the user's left eye.

As described herein, the headset 2 (or head-mounted device (HMD)) is eye glasses that are being worn by the user 1. In one embodiment, the headset may be any type of device that may be worn on a user's head, such as an eyepatch, goggles, a visor, headgear, headphones, or otherwise. In another embodiment, the headset may be any type of electronic device, which may or may not be a part of (or worn on) the user's head. For example, the headset may be a part of a user's appeal (e.g., a part of or integrated into a hat worn by the user 1). Although the headset 2 is illustrated as a single contiguous frame 4, in other embodiments, headset may be segmented into two or more body-wearable modular components that may be interconnected and mounted or worn in various locations about the body or clothing.

The headset 2 includes a camera 6, an antenna mount 5 to which one or more antennas 10 are coupled, and an input device 11 (which may include a user interface). In particular, each or least some of the elements of the headset may be coupled to the frame 4 of the headset. In this example, the frame is a glasses frame, where the elements are a part of (or integrated into) the frame. In one embodiment, the headset may include more or less elements, such as having more or less cameras.

In one embodiment, the camera may be designed to capture optical images as image data, where the data produced by each camera includes a scene of a visual representation of a field of view (FOV) of the camera. In another embodiment, the camera 6 may be designed to capture a video stream (video data), where the stream may include a series of still images (e.g., as video frames) that may be captured by the camera. In some embodiments, the camera 6 may have a FOV that is directed away from the user 1, in a forward direction with respect to the user. In one embodiment, the camera may be designed to capture color images, which may be presented onto the user's retina, as described herein. In another embodiment, the camera may be any type of image capturing device that may capture other types of images. More about the camera is describe herein. In another embodiment, the headset may include another camera that has a different FOV, which may be directed towards at least a portion of the user, such as being directed towards the user's right eye. Such a camera may be utilized to detect eye gestures/gaze of the user. More about eye gestures and eye gaze are described herein.

The antenna mount 5 is shown as being coupled to (e.g., a temple of) the frame 4, and includes one or more antennas 10. In particular, the antennas may be attached to the mount via an articulable arm, which may be user (or automatically) manipulated in order to reposition the antennas. For instance, the antennas may be repositioned such that they are within a threshold distance of the implant 3, while the implant is inside the eye 30. In one embodiment, the mount may be coupled to a different location on the frame 4. As described herein, the antennas may be configured to provide wireless communication and/or power between the headset 2 and the implant 3. In another embodiment, the headset 2 may have a wired connection (e.g., via one or more wires) to the implant (e.g., where the wires are surgically implanted through a portion (e.g., the eye) of the user.

In one embodiment, the input device 11 may be any type of device that includes one or more user interfaces that allows the headset 2 to receive user input. For example, input device 11 may be (or include) one or more physical buttons. In another embodiment, the input device may be configured to receive user input via virtual buttons, such as user interface (UI) items that are displayed on a graphical user interface (GUI) of a touch-sensitive display screen, which may be a part of the headset. More about the input device is described herein.

Turning to FIG. 3, this figure shows a cross section of the headset 2 and the (e.g., right) eye 30 of the user 1, showing the implant 3 inside the eye 30. As shown, the eye 30 includes a cornea 32 to which (or adjacent to which) the implant 3 is disposed within the eye 30, and the eye includes a retina 31. As described herein, the user's vision may be impaired due to vascularization or damage to the cornea 32, whereas the retina 31 may be capable (or at least partially capable) of converting light into electrical signals used by the user's brain to create images. In this case, the implant may be designed to output (or create) light, which may then be captured by the retina. For example, the implant includes an image formation device 33 that may be configured to reproduce one or more images (video), which may be captured by one or more cameras of the system 20, and that are directed towards (as shown by a dashed line with an arrow terminating at) the user's retina 31. Specifically, the implant 3 may receive the one or more images captured by the camera 6 through the antenna(s) 10, and may display through the image formation device. In one embodiment, the reception, decoding, and display of the image data may be executed in real-time, and provide the user with virtual, real-time, forward facing vision. More about presenting images through the use of the image formation device is described herein.

Furthermore, although FIGS. 1-3 illustrate a monocular intraocular system, the illustrated components may be replicated to implement a binocular intraocular system. Furthermore, implant 3 may be operated with different external hardware having different functionality than described herein in connection with headset 2. In fact, implant 3 may be operated without a headset, but rather receive wireless communications from a variety of sources to display a variety of different information.

As illustrated, implant 3 is entirely disposed within eye 30 and does not include electronic cables or tethers extending out of eye 30 to the headset 2. Thus, the implant may be small enough (e.g., and shaped) to fit entirely or partially inside the volume of the lens capsule of the eye, or into the anterior chamber of the eye, or into the vitreous humor of the eye. Similarly, the headset 2 may be an independent, discrete unit that is worn on the user's head. In one embodiment, the headset includes embedded electronics for powering and orchestrating the operation of intraocular system 20 including itself and implant 3.

In the illustrated embodiment, the implant 3 includes an enclosure 25 that is arranged to house (at least some of the) elements (electronic components) of the implant. In one embodiment, the enclosure 25 may be a biocompatible enclosure that is sized and shaped for implantation into eye 30. In one embodiment, enclosure 39 may be sized for implantation into the region of the capsular sack of eye 30, as described herein. In one embodiment, enclosure is a hermetically sealed enclosure fabricated of metal, polymers, or otherwise.

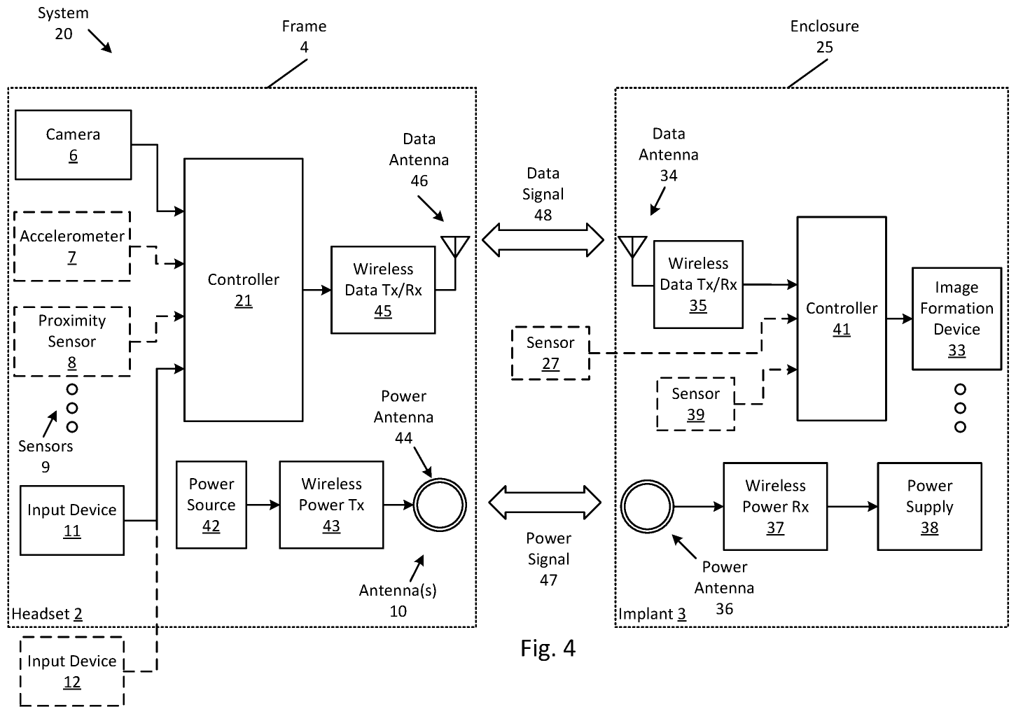

FIG. 4 shows a block diagram of the example intraocular system 20 that includes the headset 2, the implant 3, an input device 12, and a sensor 27. In one embodiment, one or more of the elements of the system may be optional, such as the input device 12 and/or the sensor 27. As described herein, while in use, the system may be worn by a user, such as the headset 2 being worn on a user's head and the implant 3 may be inside one of the user's eyes; and the headset may produce a video stream that is transmitted to the implant for display by the implant's image formation device 33. As described herein, the system may include one or more implants. For example, the user may include two implants in one eye, and/or may include at least one implant in each of the user's eyes. In either case, at least some of the operations performed by the system described herein may be performed for each of multiple implants.

As shown, the headset 2 includes several sensors 9, which include the camera 6, an accelerometer 7, and a proximity sensor 8. In one embodiment, the headset may include more or less (similar or different) sensors, such as having two or more cameras, each camera having a different FOV, as described herein. The headset 2 also includes the input device 11, a controller 21, a power source 42, a wireless data transceiver (Tx/Rx) 45, a wireless power transmitter (Tx) 43, and the antennas 10, which include a power antenna 44 and a data antenna 46. In one embodiment, each of these elements may be a part of or integrated within the frame 4 of the headset 2. In another embodiment, one or more of the elements may be separate from the (frame 4 of the) headset 2. For example, the headset may receive video data from one or more cameras that are a part of separate electronic devices (where the data may be received via a wireless data connection).

Also shown, the implant 3 includes a data antenna 34, a wireless data Tx/Rx 35, a controller 41, an image formation device 33, a power antenna 36, a wireless power Rx 37, and a power supply 38. In one embodiment, the headset and/or implant may include more or less elements, such as the headset 2 having two or more cameras and/or the implant having two or more image formation devices 33.

In one embodiment, the one or more sensors 9 are configured to detect the environment (e.g., in which the headset is located) and produce sensor data based on the environment. In some embodiments, the controller may be configured to perform operations based on the sensor data produced by one or more sensors 9, as described herein.

In one embodiment, the camera 6 may be a complementary metal-oxide-semiconductor (CMOS) image sensor that is capable of capturing digital (e.g., still) images including image data that represent a FOV of the camera 6, where the field of view includes a scene (e.g., visual representation) of an environment in which the headset 2 is located. In some embodiments, the camera may be a charged-coupled device (CCD) camera type. The camera is configured to capture a video stream (video data), which may be represented as a series of still digital images (or image frames). In one embodiment, the camera may be positioned anywhere about the headset. In some embodiments, the headset may include multiple cameras (e.g., where each camera may have a different FOV with respect to other cameras). In one embodiment, the video stream captured by the camera 6 may be high definition (HD) video that may include 10-bit 4 k video, such as, for example, of resolution 3840×2160 pixels (which is also referred to as 2160p), 1920×1080 pixels (also referred to as 1080p video), and 1280×720 pixels (also referred to as 720p video) with a frame rate of 59.94 and/or 60 image frames per second (fps). In another embodiment, the resolution and/or frame rate (as fps) of the video stream captured by the camera may be different, as described herein.

In one embodiment, the camera 6 may include one or more optical components (optics), such as one or more lenses which may be used to provide optical zoom. In another embodiment, the camera may include (or may be coupleable to) one or more optical filters, such as a polarization filter. In one embodiment, a polarization filter may help reduce or eliminate glare off of one or more objects that are captured within the scene of the video stream. As another example, the camera may include one or more multi-spectrum filters, which may be arranged to separate (or block) one or more wavelengths of light such that the camera is more sensitive to particular (select) wavelengths of light. In one embodiment, the optical filters may be physical filters.

In another embodiment, in addition to (or in lieu of) optical filters, the system 20 may include one or more digital filters, which may be applied (e.g., by the controller 21) as one or more video processing operations. For instance, standard optical cameras may use one or more wavelength filters, such as red, green, and blue filters, designed to mimic the red/green/blue receptors of a human eye. In one embodiment, the camera 6 that produces video data presented by the image formation device 33 may use different and/or more filters, to assist the user to see colors, or colored objects, in ways not possible with the naked eye (e.g., an eye that does not include an implant). In which case, the controller may perform one or more video processing operations to filter out and/or add one or more colors to one or more optical images captured by the camera 6.

In one embodiment, the accelerometer 7 may be configured to monitor motion (e.g., as a motion sensor) and produce motion data that indicates movement of the (e.g., user who is wearing the) headset 2. In one embodiment, the accelerometer may be (e.g., a part of) an inertial measurement unit (IMU) that is designed to measure (e.g., changes in) the position and/or orientation of the headset 2. In some embodiments, the proximity sensor may be any type of electronic device (e.g., an optical sensor) that is designed to detect the proximity (e.g., distance of) one or more objects within the environment, with respect to the headset.

As described herein, the system 20 may include other types of sensors. For example, the headset may include one or more microphones. In one embodiment, the headset may include any type of microphone (e.g., a differential pressure gradient micro-electro-mechanical system (MEMS) microphone) that is configured to convert acoustical energy caused by sound wave propagating in an acoustic environment into an input microphone signal.

In some embodiments, the headset may include different types of sensors, such as having different types of cameras. For example, the camera 6 may be a visible imaging sensor that is designed to collect visible light and produce color images. In one embodiment, the headset may include a thermal imaging camera (or thermographic camera) that is designed to create a thermal image using infrared (IR) radiation. A thermal image may provide a visualization of a heat signature of a scene of the environment within the FOV of the thermal camera. For example, the heat signature may be a multi-colored heat map that displays cooler objects as one color (e.g., blue), while displaying warmer objects as another color (e.g., red). As another example, the headset may include a depth camera that is designed to determine distances of objects within the camera's FOV. In one embodiment, the depth camera may produce video data that includes depth information (e.g., color coded) of objects within the video. In one embodiment, the depth information may be provided to users for display on the implant 3 who have difficulty with depth perception (e.g., because they only have one eye and therefore lack stereoscopic vision).

As described herein, the system may include an input device 12, which may be any type of electronic device that allows for user interface that is arranged to receive user input, and provide the user input to the (e.g., controller 21 of the) headset. For example, the input device 12 may be a user device, such as a smart phone, a tablet computer, a laptop computer, etc. In one embodiment, user input may be received via a physical interface (e.g., a button) or a virtual interface (e.g., a GUI displayed on a display) of the input device. Other examples of input devices may include wearable electronic devices, such as a smart watch or a haptic (virtual reality (VR)) glove, etc. In the case of a haptic glove, the input may be received through a user interface of the glove (e.g., a physical button). In another embodiment, the input may be based on a hand gesture, while the glove is worn by the user. For instance, the glove may include one or more sensors, which may detect user input based on hand (e.g., finger) movements. In one embodiment, the input device may be a wireless device that may be communicatively coupled to the headset. For instance, the input device 12 may be configured to establish the wireless connection (e.g., via the wireless data Tx/RX) with the headset via a wireless communication protocol (e.g., BLUETOOTH protocol or any other wireless communication protocol). During the established wireless connection, the input device may exchange (e.g., transmit and receive) data packets (e.g., Internet Protocol (IP) packets) with the headset, which may include user input data. In another embodiment, the input device 12 may be connected to the headset 2 via a wired connection.

The controller 21 (and/or the controller 41 of the implant) may be a special-purpose processor such as an application-specific integrated circuit (ASIC), a general purpose microprocessor, a field-programmable gate array (FPGA), a digital signal controller, or a set of hardware logic structures (e.g., filters, arithmetic logic units, and dedicated state machines). In one embodiment, the controller may include memory which may store one or more instructions, which when executed by the controller causes the headset to perform at least some of the operations described herein. In another embodiment, the controller 21 may be configured to receive sensor data produced by one or more sensors 9, receive user input from one or more input devices, perform video signal processing operations (or video processing operations), and/or networking operations, as described herein. In particular, the controller may be configured to determine whether video data captured by one or more cameras (and for presentation to the user through the implant) should be enhanced (e.g., augmented, modified or adjusted) based on sensor data, for example. In particular, the controller may perform one or more video processing operations to enhance video captured by the camera 6, and transmit the enhanced video to the implant for display on the image formation device 33. More about the operations performed by the controller 21 is described herein.

In one embodiment, the controller 21 may perform one or more video processing operations upon video data captured by the camera. In particular, the controller may perform a brightness enhancement, a contrast enhancement, and/or a color enhancement. For example, scene that is being displayed to the user (through the image formation device 33) may be contrast-enhanced to increase contrast and improve visibility of objects within the scene. As described herein, the controller may perform such enhancements based on user input (e.g., based on a user adjusting optical characteristics of the video, such as a contrast control, etc.) and/or based on sensor data. In another embodiment, the controller may perform a video processing operation upon video data based on one or more characteristics of the video data. For example, the controller may perform a brightness enhancement to reduce the brightness when the video is too bright (e.g., above a brightness threshold). More about performing video processing operations are described herein.

In another embodiment, the controller may be configured to augment video data by superimposing information onto at least a portion (or area) of the video that is to be displayed to the user. In particular, the controller may augment a visual representation of a scene of the environment in a video stream captured by the camera, such that information (e.g., text, images, video, etc.) is superimposed above (overlaid on top of) a portion of the scene. In which case, the intraocular system may operate as an augmented reality (AR) system. In one embodiment, to augment the video, the controller may receive (produce) data (e.g., an image, text, video), and may alpha blend the data with the video stream captured by the camera 6. For example, the controller 21 may produce a separate video stream based on the received data, and may alpha blend the separate video stream with the received (captured) video stream to produce an augmented video stream. In another embodiment, the controller may use any video processing operation to superimpose information onto the video (image) data.

In one embodiment, the video stream may be augmented with information or data from one or more data sources. In particular, the video stream may be augmented with sensor data captured by one or more sensors 9. For example, motion data captured by the accelerometer 7 may be added in order to notify the user of a direction of movement. More about augmenting the video based on sensor data is described herein. In one embodiment, information may be received from one or more application programs that are being executed by (e.g., the controller 21 of) the headset. For example, the controller may be executing a navigation application that produces navigation information, such as driving instructions, from a starting location to a destination. In which case, the controller may be configured to superimpose the driving instructions onto the video stream, such that when presented to the user, the user may view the instructions overlaid on top of a scene of the environment. In another embodiment, the information or data may be received from one or more electronic devices that are communicatively coupled with the controller 21. For instance, the headset may be coupled to a user device, such as a smart phone that is executing the navigation application. In which case, the headset may receive navigation instructions from the user device for augmenting the video stream.

As described thus far, the intraocular system may operate as an AR system, which augments a video stream captured by the camera 6 of the headset that includes a scene of an environment of the user (e.g., a FOV projecting in front of and away from the user) with information or data. In another embodiment, the controller 21 may (present and/or) augment video data of a virtual environment. In which case, the intraocular system may operate as a virtual reality (VR) system that presents a virtual environment in which the user of the system may participate. For example, the controller 21 may receive a virtual reality video stream that includes a scene of a virtual environment (e.g., from a FOV of an avatar within the virtual environment). In one embodiment, the user may interact with the virtual environment through one or more input devices. For instance, the input device 12 may be one or more peripheral devices, such as a mouse and a keyboard, with which the user may use to interact. In one embodiment, while viewing (and interacting) with a VR environment, the implant may not display (or only partially display) video data captured by one or more cameras of the headset 2.

As described thus far, the controller may be configured to augment, or video enhance a video stream for display to the user. In one embodiment, the controller may perform one or more of these operations to enhance the video stream. For example, the controller may perform a contrast enhancement and augment information onto a captured video stream. For example, as described herein, the headset may include a depth camera that includes depth information. In which case, the controller may be configured to produce a depth map of a scene of the environment captured by the depth camera, where the controller applies different colors within the scene to indicate a depth (distance) to objects within the scene. In addition or in lieu of the color coordination, the controller may augment the scene may adding text that indicates a distance between the depth camera and the object within the environment.

In one embodiment, the controller 21 may be configure to format (encode) video data for transmission to the implant. For instance, the controller may receive video data from the camera 6 and encode the video data into a format that may be suitable for wireless transmission and/or for presenting (e.g., displaying) by the (e.g., image formation device 33 of the) implant. For example, the controller may encode the video data into a desired format, such as MPEG-4, and provide the video data to the wireless data Tx/Rx 45, which may process the video data for transmission. For example, the data Tx/Rx 45 may use any number of encoding techniques including one or more of frequency modulation, phase modulation, amplitude modulation, and/or time multiplexing. The headset may transmit the encoded data as a data signal 48 (e.g., a RF signal) via the data antenna 46 to the implant 3. In one embodiment, the data signal 48 may be any type of wireless connection, such as BLUETOOTH.

In one embodiment, the implant 3 may receive a video stream (via the data signal 48) and use the image formation device 33 to present the video stream. In particular, the wireless data Tx/Rx 35 of the implant may receive the data signal 48 via the data antenna 34, and may extract the video stream from the received signal 48, and provide the video stream to the controller 41. The controller 41 may be configured to process (e.g., decode) the video stream, and present the video stream through the image formation device 33. More about the controller 41 is described herein.

In one embodiment, the image formation device 33 may be any type of electronic device that projects light onto at least a portion of the user's retina. For example, the device may include a laser projection system that has a laser projector that is configured to produce a laser beam (e.g., using a light source, such as a laser diode) that is projected onto a spot on the user's retina to reproduce image data. In one embodiment, the laser projector may be a scanning projection system that may include one or more (scanning) mirrors, one or more lens, and one or more actuators, optics (e.g., having an adjustable prism, one or more lens, etc.). Such a scanning projection system may be arranged to direct the laser beam onto the retina by using the actuators to actuate at least one of the mirrors. In particular, the system actuates the mirrors to scan the retina with the laser beam by moving across the retina generally horizontally in one dimension and/or generally vertically in another dimension.

In another embodiment, the image formation device may include a micro-display. In one embodiment, micro-display may be implemented as a (e.g., two-dimensional (2D)) multi-color light emitting diode (LED) display array. In other embodiments, micro-display may be a backlit liquid crystal display (LCD), a monochrome LED display array, an organic LED (OLED) display, or otherwise. In one embodiment, the micro-display may have a resolution of between 30,000 pixels to 6 million pixels. In another embodiment, the resolution may be between 40,000 pixels and 4 million pixels. Examples of resolutions for a micro-display with an aspect ratio of 16:9 include 256×144, 1,280×720, 1,920×1,080, and 2,560×1,440. In another embodiment, the micro-display may have any aspect ratio and/or any resolution. In one embodiment, micro-display may be a 5 mm diameter display while enclosure 25 of the implant may have an overall 10 mm×10 mm. In one embodiment, the micro-display outputs the image based upon the received image data. In one embodiment, the image formation device may include optics (e.g., one or more lens and/or mirrors), which may be used to focus the light projected by the micro-display onto retina 31.

As described herein, the image formation device 33 may include adjustable components that provides an adjustable focal distance (e.g., z-axis adjustment) to a regenerated image. In various embodiments, the formation device further include one or more adjustable prisms that provide beam steering for lateral adjustments (x and/or y axis adjustment) of the position of the projected image, as described herein. Lateral adjustments ensure that a projected image is properly positioned/centered on retina 31 including the user's fovea. In one embodiment, the image formation device may display a video stream as a series of one or more regenerated (or projected) images as output by the image formation device.

In one embodiment, the implant 3 may include a sensor 39 that is arranged to provide sensor data to the controller. In one embodiment, the sensor 39 may include a temperature sensor to monitor the operational temperature of the implant. In this regard, the temperature sensor is a proxy reading for power consumption or power dissipation within implant. The temperature sensor also serves as a safety measure to ensure the eye tissue surrounding implant is not damaged due to elevated operational temperatures.

In one embodiment, the sensor 39 may include a voltage sensor coupled to the power supply 38 to measure and monitor the voltage across the supply, and thus measure the stored energy (e.g., in one or more power storage units of the supply). The measured voltage across power supply 38 may also serve as a proxy for, or an indication of, the reception strength of power signal 47.

Returning to the headset 2, the power source 42 may be any type of power source that is capable of powering (e.g., supplying current) to electronics of the headset 2. For instance, the power source may be a rechargeable battery that is housed within the headset 2. In one embodiment, the battery may be removeable such that it may be charged. As described herein the power source may also be capable of supplying power to operate the (e.g., electronics of the) implant 3. Specifically, the wireless power Tx 43 is configured to draw power form the power source 42 and wirelessly transmit power as a power signal 47 via the power antenna 44 to the implant. In particular, the power source 42 may provide power via inductive power transfer. For instance, the wireless power Tx 43 may produce the power signal 47 as an electromagnetic filed using the power antenna 44. The wireless power Rx 37 converts the electromagnetic field, via the power antenna 36 into a current that is supplied to the power supply 38. In another embodiment, the power signal 47 may be any type of signal, such as an optical signal, radio frequency (RF), infrared (IR), etc., that is capable of wirelessly transmitting (over the air) power over a distance. In one embodiment, the power supply may include a power storage device (e.g., battery) to store the received power, and enables for continuous or uninterrupted supply of power to the electronics (e.g., the controller 41 and the image formation device 33) of the implant. In another embodiment, the power supply may condition and provide power to the implant, while the headset is within a threshold distance of the implant (e.g., while worn by the user). If, however, the headset is removed from the user's head, the power signal 47 may no longer be received by the implant, and as a result the implant may stop operating, thereby deactivating (ceasing to power) the image formation device 33.

As described thus far, the controller 41 may be configured to receive video data, via the data signal 48, from the headset 2, and display the video data on the image formation device 33. In one embodiment, the controller 41 may be configured to receive sensor data (e.g., via a wired or wireless connection) from one or more sensors, such as sensor 27, which may be disposed within (or adjacent to) the user's eye. In one embodiment, the sensor 27 may be an electronic device (e.g., an electromyogram (EMG) sensor) that is surgically implanted within (or adjacent to) the user's eye, and may be configured to produce an electrical signal based on electrical activity from muscle movement. For example, the sensor 27 may be arranged to sense eye gestures (e.g., by detecting muscle movement of the orbicularis oculi), and produce sensor data that indicates that the user has performed the eye gesture. More about the sensor 27 and the controller 41 are described herein.

As described herein, the controller 21 of the headset 21 may be configured to process one or more video streams and transmit the video streams to the controller 41 of the implant, where the controller 41 may be configured to display the video stream on the image formation device. In another embodiment, either of the controllers may perform at least some of the operations described herein. For example, the controller 41 may be configured to receive the video stream from the headset, and process the video stream by performing one or more video signal processing operations. More about the operations performed by the controllers are described herein.

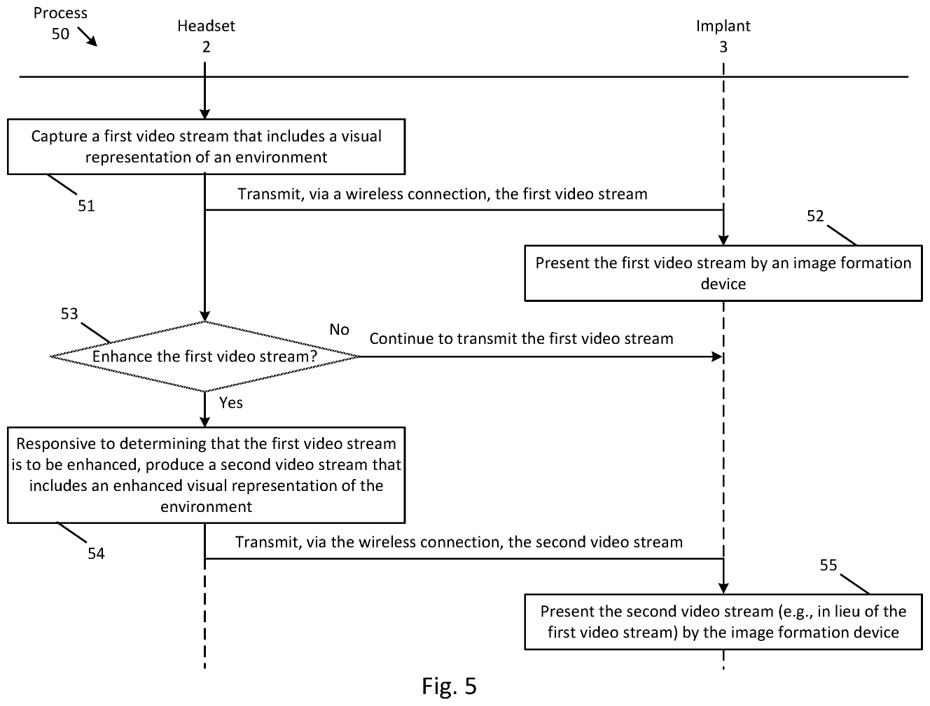

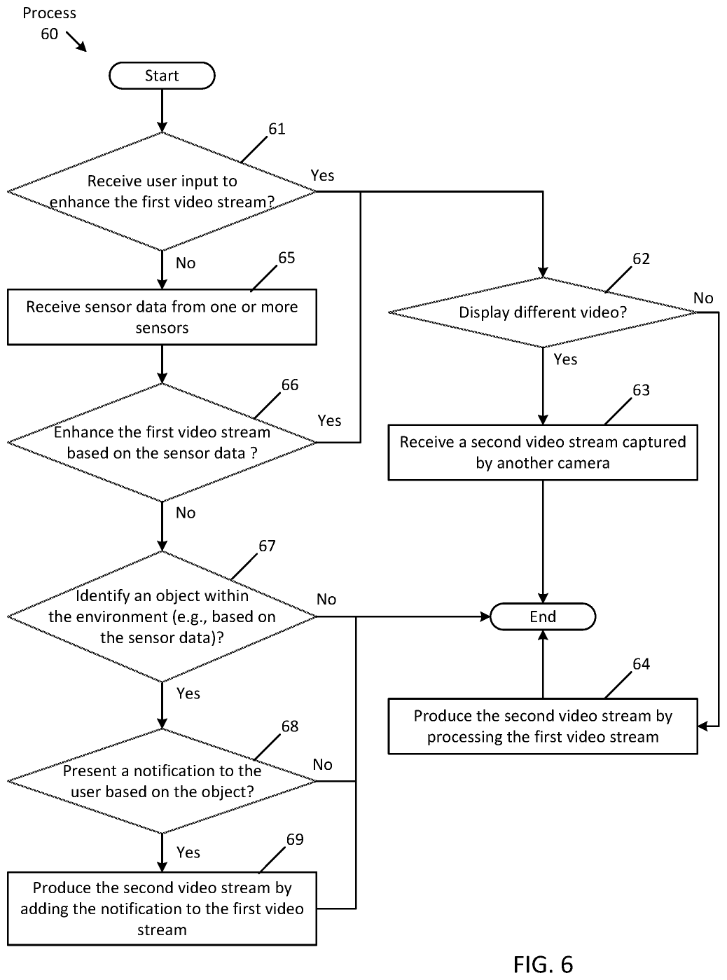

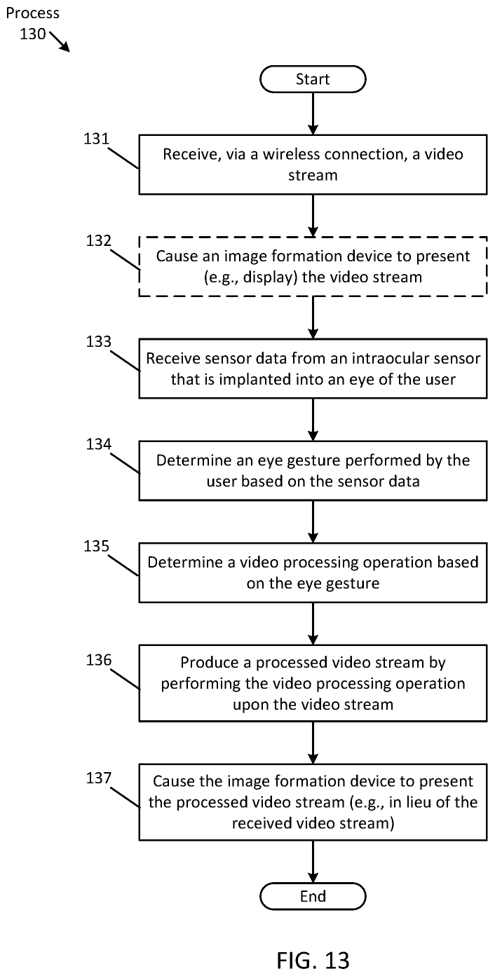

FIGS. 5, 7, and 10 are signal diagrams that include processes 50, 70, and 100, respectively that include operations that are performed by the headset 2 and the implant 3. In particular, at least some operations may be performed by the controller 21 of the headset 2 and at least some operations may be performed by the controller 41 of the implant. FIGS. 6, 8, 9, 11, and 12 are flowcharts that include processes 60, 80, 90, 110, and 120, respectively, that include operations that are performed by (e.g., the controller 21 of) the headset 2, and FIG. 13 is a flowchart that includes process 130 that includes operations performed by (e.g., controller 41 of) the implant 3. In one embodiment, although the operations described herein may be with respect to a particular device (e.g., process 60 of FIG. 6 being performed by the headset), any of the operations described herein may be performed by one or more devices. For example, operations described herein that are performed by the headset may at least partially be performed by the (e.g., controller 41 of the) implant.

Turning now to FIG. 5, this figure is a signal diagram of one embodiment of a process 50 that is performed by (e.g., the headset 2 and/or the implant 3 of) the example intraocular system 20 to enhance a visual scene that is projected onto a user's retina. In particular, a visual representation may be “enhanced” such that the user views an enhanced visual scene which may include additional (e.g., augmented) information and/or different information in addition to or in lieu of a (e.g., color) visual scene that is captured by the camera 6 of the headset 2. For example, the video stream may be enhanced by adding descriptive information (e.g., superimposed) over the scene of the visual representation. As another example, the video stream may be enhanced to improve visibility of the scene by reducing or eliminating aspects of the scene that would otherwise adversely affect a visual experience (e.g., adjusting the brightness of the scene due to the scene being too bright or too dark). As a result, the user may view an environment differently than a person who does not include an intraocular system (e.g., an implant inside the person's eye) and/or a person with “normal” vision (e.g., having non-opaque corneas with fully functional retinas).

The process 50 begins by the controller 21 of the headset 2 capturing a first video stream that includes a visual representation (as a scene) of an environment of the headset 2 (at block 51). In particular, the camera 6 of the headset 2 that is being worn by the user 1 may capture the first video stream, where the video stream may be a collection of color images (e.g., as video frames) that are captured in series. The controller 21 transmits, via a wireless connection, the first video stream (e.g., as the data signal 48) to the implant 3. In one embodiment, the wireless connection may already be established between the devices. In another embodiment, the headset 2 may establish the connection once the first video stream is captured. In which case, the controller 21 may be configured to transmit a control (e.g., handshake) signal, via the data signal 48, to the implant 3 for establishing a connection. In response, the implant 3 may transmit an acknowledgment signal to set up the wireless connection. In one embodiment, the system may use any type of wireless protocol for establishing the wireless connection.

The controller 41 of the implant 3 may receive the first video stream (e.g., via the wireless data transceiver 35), and may present the first video stream by the image formation device 33 (at block 52). For instance, the controller may render the first video stream onto a micro-display of the image formation device 33, such that pixel light output of a 2D array of pixels of the micro-display is projected towards and onto at least a portion of the user's retina.

Returning to the headset 2, the controller 21 (e.g., while the first video stream is being captured and transmitted to the implant 3) determines whether the first video stream is to be enhanced (at decision block 53). In one embodiment, the determination may be based on whether the controller 21 has received user input through one or more input devices. For example, the controller may determine that the video is to be enhanced based on the user of the intraocular system selecting one or more selectable (e.g., UI) items of a user interface (e.g., GUI) of the input device 12. In another embodiment, the determination may be based on sensor data from one or more sensors 9 of the headset and/or sensor data from the sensor 27. For example, the controller 21 may determine to perform one or more video processing operations upon the video stream based on one or more (e.g., optical) characteristics of the first video stream. As an example, when the first video stream has a brightness (e.g., across at least a portion of the scene captured by the camera 6) is greater than a threshold (e.g., generally too bright for a person to look at), the controller 21 may determine that the first video stream is to be processed to a lesser brightness level. More about how the first video stream is determined to be enhanced is described herein.

Responsive to determining that the first video stream is to be enhanced, the controller 21 produces a second video stream that includes an enhanced visual representation of the environment (at block 54). Continuing with the previous example, the controller may perform a brightness adjustment to the first video stream to reduce the brightness (e.g., below a threshold). Thus, in this example, to produce the second video stream the controller may process (modify or adjust) the first video stream by applying one or more video processing operations, for example. In another embodiment, the controller may produce a second video stream based on image (or video) data captured by one or more cameras (e.g., other than camera 6) of the intraocular system. More about producing the second video stream is described herein.

The controller 21 transmits, via the wireless connection, the second video stream to the (e.g., controller 41 of the) implant 3 that is inside the eye of the user, where the implant may be configured to use the image formation device to present the second video stream. In particular, the controller 41 presents the second video stream (e.g., in lieu of the first video stream) by the image formation device (at block 55). In one embodiment, the controller may transition between the first video stream and the second video stream in a seamless fashion. In which case, the controller may be configured to cross-fade between the two video streams. In one embodiment, the controller may notify the user (e.g., via a pop-up notification displayed on the image formation device 33) that a transition between the video streams is going to (or has already) occurred.

In one embodiment, the intraocular system will transition between the video streams automatically (e.g., without user intervention). In another embodiment, the intraocular system may request user authorization from the user to transition from the first video stream to the second video stream. In which case, the image formation device may present a notification, where in response to receiving user input (e.g., through input device 11), the system may present the second video stream.

FIG. 6 is a flowchart of one embodiment of a process 60 to determine whether to enhance captured video for presentation by the implant 3. In particular, at least some of the operations of process 60 may be performed by the controller 21 at decision block 53 and/or block 54 of process 50 in FIG. 5.

The process 60 begins by the controller 21 determining whether user input has been received to enhance the first video stream that is being presented to the user through the image formation device 33 (at decision block 61). In particular, the controller may determine whether user input has been received from one or more input devices (e.g., devices 11 and/or 12) indicating a user desire to view enhanced video. For example, the user may select a user interface of device 11 to enhance and/or augment the video data that the user is currently viewing, such as adjusting the brightness or adding information of a software application program that is being executed by the system 20, such as adding navigation instructions from a navigation application. As another example, the controller 21 may receive an indication from input device 12 that the video stream that is being captured by the camera 6 is to be augmented with information. For example, the user may initiate a navigation application on the input device 12, which in response may transmit navigation instructions to the controller to be superimposed onto video that is being viewed by the user. As another example, the controller 21 may receive a voice command spoken by the user that includes a request to enhance the video (e.g., the user saying “Enhance brightness by 20%”). In particular, the sensors 9 may include a microphone that captures speech of the user as a microphone signal. The controller 21 may perform a speech (voice) recognition algorithm upon the microphone signal to determine whether the speech includes a voice command, as illustrated above.

If user input is received to enhance the first video stream, the controller 21 determines whether to display different video (at decision block 62). In particular, the controller determines whether the user input indicates that the user desires to view video captured by a camera other than the camera that is capturing the first video stream (e.g., camera 6). For example, the user interface of the input device may include buttons (e.g., UI items) that allow the user to select which between cameras of the (e.g., headset 2 of the) system the user wishes to view. For example, the headset may include another camera with a different FOV, and/or may include another type of camera, such as a thermal imaging camera, which may produce video data that includes one or more thermal images with a heat signature of (at least a portion of) a scene of the environment. If the user input indicates that different video is to be displayed, the controller 21 receives a second video stream captured by another camera (e.g., of the headset) (at block 63). For instance, the user input may indicate that the user wishes to view a heat signature of the scene of the environment. In which case, the controller 21 may receive video captured by the thermal imaging camera, and may transmit the video to the implant for display to the user. As another example, the user input may indicate that the user wishes to view the scene in night vision. In response, the controller may be configured to capture infrared (IR) imaging to produce a low-light (e.g., version of the first) video stream. For instance, the controller may activate one or more IR emitters (e.g., near-IR with wavelengths between 750-1,000 nm), which may produce IR light that is captured by one or more cameras to produce low-light video data.

If, however, the system is not to display different video (or is to continue to at least partially display the first video stream), the controller produces the second video stream by processing (e.g., augmenting) the first video stream (at block 64). In one embodiment, the controller may apply one or more video signal processing operations upon the video stream, such as a brightness enhancement, a contrast enhancement, a color enhancement, etc., which may be based on the user input. As an example, the controller may adjust the brightness of the first video stream that is being displayed to the user based on user input requesting an increase in brightness (e.g., the user's voice command requesting an increase in brightness by 20%, as previously illustrated). In another embodiment, the controller may apply one or more optical filters upon the first video stream. For instance, the controller may cause the camera 6 to use one or more filters, such as a polarization filter, a color filter, etc., while capturing the first video stream. In another embodiment, the controller may digitally apply one or more of the filters (e.g., by applying one or more color filters upon the first video stream.

In another embodiment, the controller 21 may augment the first video stream to include information or data, as described herein. For instance, the user input may indicate a desire to view sensor data, such as temperature data of the environment. In which case, the controller may retrieve temperature data from a temperature sensor of the headset, and overlay the temperature data on top of (at least a portion of) the first video stream, such as superimposing the temperature data on a top coroner of a scene captured within the first video stream.

Returning to decision block 61, if no user input is received to enhance the first video stream, the controller 21 receives sensor data from one or more sensors (at block 65). In particular, the controller 21 may receive accelerometer data from the accelerometer 7, may receive video/image data from one or more cameras, may receive proximity data from the proximity sensor 8, etc. In one embodiment, the controller 21 may receive sensor data from sensors 9 and/or from sensors that are separate from the headset (e.g., via a wireless connection). For example, the controller may receive video data from a camera that is a part of an electronic device, such as the input device 12.

The controller 21 determines whether to enhance the first video stream (e.g., that is being presented to the user through the image formation device 33 of the implant 3) based on the sensor data (at decision block 66). In particular, the controller 21 monitors the sensor data, which may include video data of the first video stream, to determine whether the video stream is to be processed. In one embodiment, the controller may monitor video data captured by one or more cameras of the system 20 to determine whether the user has made a gesture indicating a request to enhance the first video stream. For instance, the controller may perform an object recognition algorithm upon the first video stream to determine whether the user is making a particular gesture, such as a hand gesture, that indicates a desire for the system 20 to enhance the video. An example of such a gesture may be covering the user's eye, indicating that the environment is too bright. As another example, the controller may determine whether the first video stream is to be enhanced based on one or more characteristics of the stream. For instance, the controller may determine whether a characteristic exceeds a threshold, such as whether a brightness level of the stream is above a brightness threshold. If so, the controller may proceed (e.g., to block 64) to process the first video stream by performing one or more video processing operations such that the characteristics remain below the threshold. In this case, the controller 21 may lower the brightness of the first video stream. Conversely, the controller may use sensor data to determine whether the brightness level is too low. For instance, the controller may monitor a light sensor of the headset (and/or the brightness of the first video stream), to determine whether the brightness is too low, which may be the case when the user has entered a dark room. If so, the controller may proceed to enhance the first video stream (e.g., at block 64) by producing the first video stream in low light conditions, such as producing a night-mode (night vision) of the first video stream. In particular, the headset may include one or more infrared (IR) LEDs that may be used with an IR sensitive camera of the headset to produce a second video stream that includes a night mode of the captured scene of the environment.

In another embodiment, the controller 21 may determine to enhance the first video stream based on whether sensor data exceeds a threshold. For instance, the controller may determine whether temperature data of a temperature sensor exceeds a temperature threshold. If so, the controller may proceed to augment the first video stream to include a temperature notification to the user.

If the first video stream is not determined to be enhanced based on the sensor data (e.g., exceeding a threshold), the controller 21 determine whether an object is within the environment based on the sensor data (at decision block 67). In which case, the controller may determine whether the first video stream may be enhanced based on whether an object is within the environment. For example, the controller may perform an object recognition algorithm upon the first video stream and/or other video streams captured by other cameras to identify an object contained therein. Examples of objects may include inanimate objects, animals, and other people. In another embodiment, the controller may determine whether an object is within the environment based on proximity sensor data (e.g., indicating that the object is within a threshold distance of the headset). In yet another embodiment, the controller may acoustically determine whether an object is within the environment. In which case, the controller may receive one or more microphone signals and perform a sound source recognition algorithm upon the microphone signals to identify a sound source within the environment.

If an object is identified within the environment, the controller determines whether to present a notification to the user based on the object (at decision block 68). In one embodiment, the controller may notify a user of the object based on the location and/or movement of an object within the environment. For instance, the controller may receive, from the proximity sensor 8, proximity data that indicates a distance that the headset is from an identified object within the environment. In another embodiment, the controller may use the proximity data to determine a speed at which an object is moving towards the headset. In which case, upon determining that the object is within a threshold distance, the controller may produce the second video stream by adding (e.g., superimposing) a notification relating to the object to (e.g., a portion of) the first video stream (at block 69). In which case, the notification may notify the user of a presence of the object within the environment and/or characteristics of the object. For instance, the notification may notify the user of the object, such as “A wall is in front of you.”. Upon determining that an object is moving towards the user, the notification may indicate a speed and trajectory of the object, such as “A bicycle is approaching you at a high rate of speed towards your right-hand side”.

In another embodiment, such an object identification and notification may provide a user with peripheral vision enhancement. For example, along with being visually impaired, the user may have a reduced peripheral field of view. In which case, the system may use sensor data to determine whether there are stationary objects and/or whether there are objects moving towards the user (from any direction), and may alert the user of such objects in order to avoid a collision. In some embodiments, the notification may indicate a direction of the object and/or characteristics of the object (e.g., a speed at which the object is moving). In some embodiments, the controller may use video data of cameras that have different FOVs, such that the peripheral vision enhancement may be applied around an area (e.g., a 360° area) the user. For example, the system may alert the user of an object that a person with normal vision would not be able to see, such as “Warning: A car is approaching you from behind!”

In another embodiment, the notification may be based on the object. In particular, the controller may apply an object recognition algorithm upon an object within the environment to identify the object, such as a name of the object, characteristics of the object, etc. In one embodiment, such an algorithm may be a machine learning (ML) model (e.g., a neural network) that may be trained to identify objects as output based on sensor data (e.g., video data) as input. Such an algorithm may identify an object and produce a notification based on the object. In one embodiment, the ML-based algorithm may output characteristics and/or related information to an identified object. For example, upon identifying poison oak, the ML-based algorithm may produce a notification alerting the user of the oak, such as superimposing a notification of “Warning! Poison oak is ahead!”. In particular, the controller may add a notification by highlighting a region surrounding the poison oak within the video stream in order to emphasis the importance (e.g., danger) of the poison oak. In another embodiment, the ML model may be trained to recognize groups of people, such as known family and friends of the user compared to strangers. For example, the ML model may be trained to perform facial recognition upon people that are identified within a captured scene by one or more cameras of the headset. In which case, the controller may display a notification to the user indicating people captured within a scene of the first video stream that are known (e.g., by displaying their name, relationship, etc.) to the user.

Some embodiments may perform variations to the process 60 described herein. For example, the specific operations of the process may not be performed in the exact order shown and described. The specific operations may not be performed in one continuous series of operations and different specific operations may be performed in different embodiments. For example, the controller may either display a different video or produce the second video by processing (e.g., augmenting) the first video, as described herein. In another embodiment, the controller may perform both operations in order to enhance video output. For instance, upon detecting a fire at decision block 66 (e.g., based on an analysis of the first video stream), the controller may be configured to receive (or produce) a heat map based on thermal image data from a thermal camera, and may also augment information onto the heat map to notify the user of the fire, such as “Warning: Fire Detected!”.

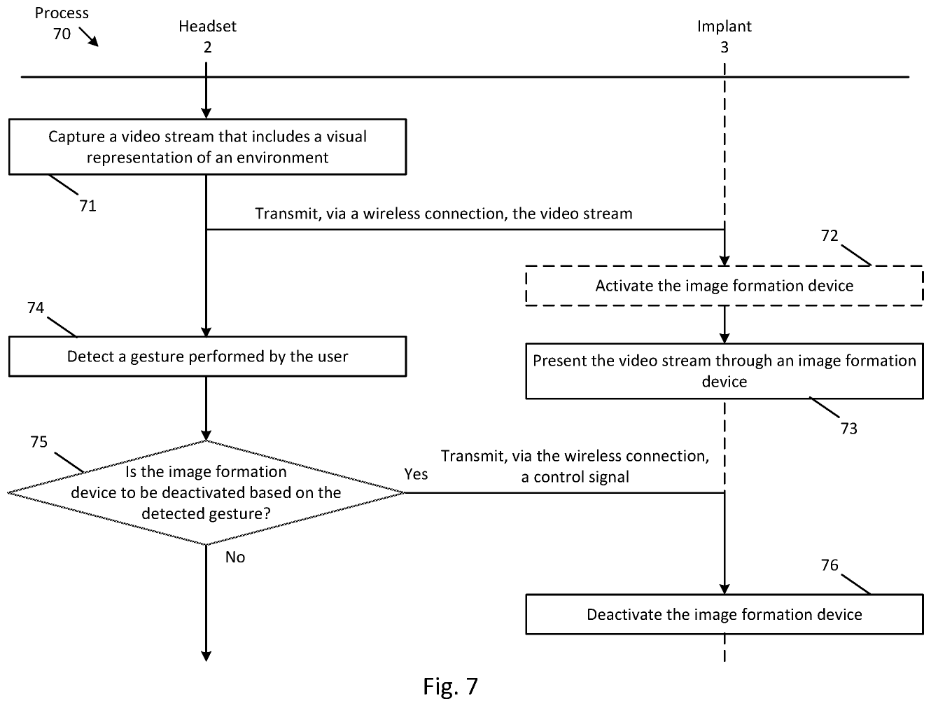

FIG. 7 is a signal diagram of one embodiment of a process 70 by the example intraocular system 20 to deactivate the image formation device 33 of the implant 3. As described herein, the image formation device may be deactivated such that the device is no longer presenting images onto the user's retina. For example, the implant may deactivate the micro-display by ceasing to display video data on the display. In one embodiment, even though the display is deactivated, the implant may be in a sleep or standby mode, thereby allowing the implant to continue to communicate with the headset. As described herein, this may allow the implant to reactivate the image formation device based on received instructions by the headset.

In one embodiment, deactivating the device has several advantages. For instance, during times at which the user sleeps, with both eyes closed, the system may deactivate the device. In addition, deactivating the image device may reduce or minimize power consumption, allowing the system to operate for longer periods of time, while the system draws power from a limited power source, such as a rechargeable battery. In addition, the device may be deactivated in order to provide the user with a more natural viewing experience. Returning to the previous example, unlike people without the implant who would view darkness while their eyes were closed, even for a short period of time, the implant may continue to provide a user with images regardless of whether the user's eyes are open or closed. As a result, the system may be configured to deactivate the image formation device during times at which a person without the implant would not see anything, such as when the user blinks, winks, covers their eyes (e.g., with a hand), and/or voluntarily (or involuntary) closes their eyes.

The process begins by the controller 21 capturing (e.g., using the camera 6 of the headset and while the headset is worn by the user) a video stream that includes a visual representation (scene) of an environment in which the headset is located (at block 71). The controller 21 transmits, via a wireless connection, the video stream to the implant that is inside the eye 30 of the user. The controller 41 of the implant 3 receives the video stream and activates the image formation device (at block 72). In particular, prior to receiving the video stream, the implant may have been deactivated. For instance, the user may have been asleep, and once the user had placed the headset on the user's head, the headset may begin capturing and transmitting video data. In one embodiment, the controller 21 may determine that the headset 2 has been placed on the user's head based on proximity data from one or more proximity sensors. Once the implant receives the video data, the image formation device may activate. In another embodiment, the operations of block 72 may be optional. In which case, the image formation device may already be active. The controller 41 presents the video stream through (or by) the image formation device 33 (at block 73).

Returning to the headset 2, the controller 21 detects a gesture performed by the user (at block 74). In one embodiment, a detected gesture may be based on user input, via the input device 11 (e.g., based on a user selecting a button of the input device 11). In another embodiment, the user input may be a voice command that is captured by one or more microphones of the intraocular system. In particular, the voice command may include one or more phrases (or words), which when recognized by a voice recognition algorithm of the controller may indicate the user's desire for the image formation device to be deactivated (when already active). In another embodiment, the user input may be received through the input device 11 of the headset 2. For example, the input device 11 may include an “on/off” button, which may allow the user to manually activate (turn on) or deactivate (turn off) the image formation device. In another embodiment, the gesture may be the user removing the headset 2 from the user's head.

In another embodiment, the gesture may be a physical gesture performed by the user, either voluntary or involuntary. In one embodiment, a voluntary gesture may be a gesture that is known to the user to cause the system to perform a particular action. In this case, the user may perform a voluntary gesture that may be used to deactivate the image formation device, such as pressing the on/off button. In another embodiment, an involuntary gesture may be a natural gesture that the user may not perform on purpose, such as blinking or saccadic eye movements, from which the system may determine that a particular action may be performed. For instance, the controller 21 may detect an eye gesture of the user's eyes closing for a period of time (e.g., due to the user going to sleep), by analyzing video data captured by a camera with a FOV that includes the user's eye(s). More about detecting gestures is described herein.

The controller 21 determines whether the image formation device 33 is to be deactivated or remain active (e.g., if already active and displaying video data, as described herein) based on the detected gesture (at decision block 75). Continuing with the previous example, the controller may determine that the image formation device 33 is to be deactivated based on the user's eye closing (e.g., for a period of time). Conversely, the controller may determine that the image formation device is to remain active to continue presenting the video stream upon determining that the gesture (e.g., remaining active while the user rubs the eye for a moment). In one embodiment, responsive to determining that the implant is to remain active, the headset 2 may continue to transmit the video stream to the implant. Responsive to determining that the image formation device is to be deactivated, the controller transmits, via the wireless connection, a controls signal (e.g., having instructions) to the implant to cause the implant to deactivate. In one embodiment, in addition to or in lieu of, transmitting the control signal, the controller 21 may cease transmitting the video stream to the implant, which may cause the implant to deactivate the image formation device. The controller 41 of the implant 3 receives the control signal, and responsive to the control signal deactivates the image formation device (at block 76). In particular, the control signal causes the image formation device to be deactivated by instructing the image formation device to cease displaying the video stream. In another embodiment, the control signal may cause the implant to enter a sleep state (mode), which may cause the implant to cease displaying the video stream while remaining at least partially powered in order to allow at least some communication with the headset.

In one embodiment, the control signal may deactivate the image formation device may putting the image device in a state at which less power may be drawn by the image formation device. For example, rather than deactivating the image formation device completely (e.g., by not providing power to the image formation device), the control signal may cause the image formation device to reduce its brightness and/or lower its frame rate, both of which may cause the image device to draw less power than during “normal” operation (e.g., while the image formation device operates at an optimal brightness and/or frame rate). As an example, the image formation device may be deactivated by causing the micro-display of the image device to display black pixels, which may provide less luminescence than if the micro-display were otherwise displaying color images.

In one embodiment, the system may perform similar operations to determine whether to activate (or reactivate) the image formation device. For example, the headset 2 may continue to monitor sensor data to detect whether the user performs another gesture that indicates that the image formation device is to be activated, such as an eye gesture of opening the closed eye. In response, the controller 21 may transmit another control signal to the implant to activate the image device. In one embodiment, in addition to transmitting the control signal, the controller 21 may continue to transmit the captured video stream for display by the image formation device once it is activated.

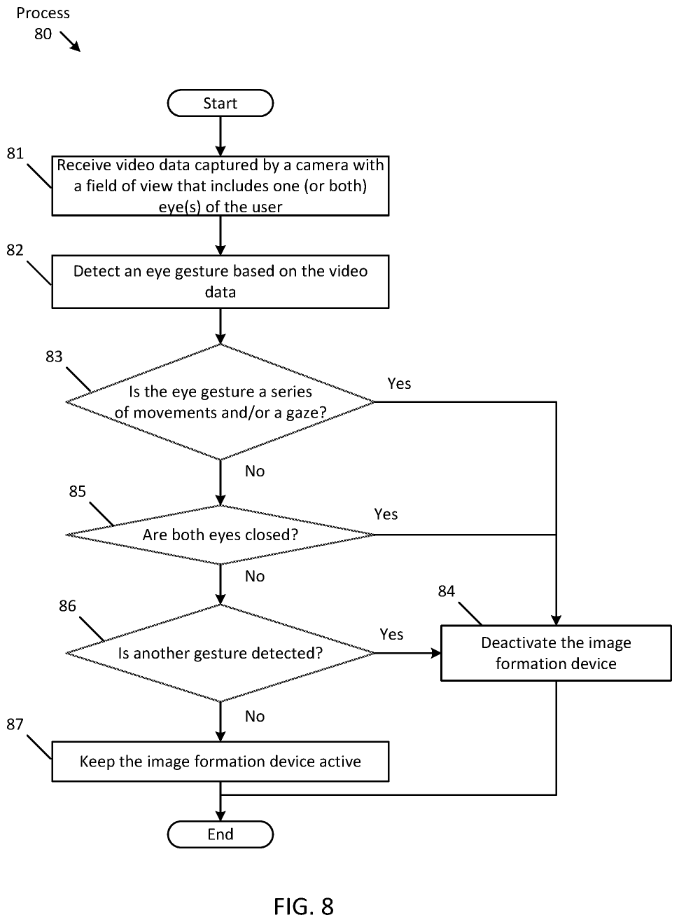

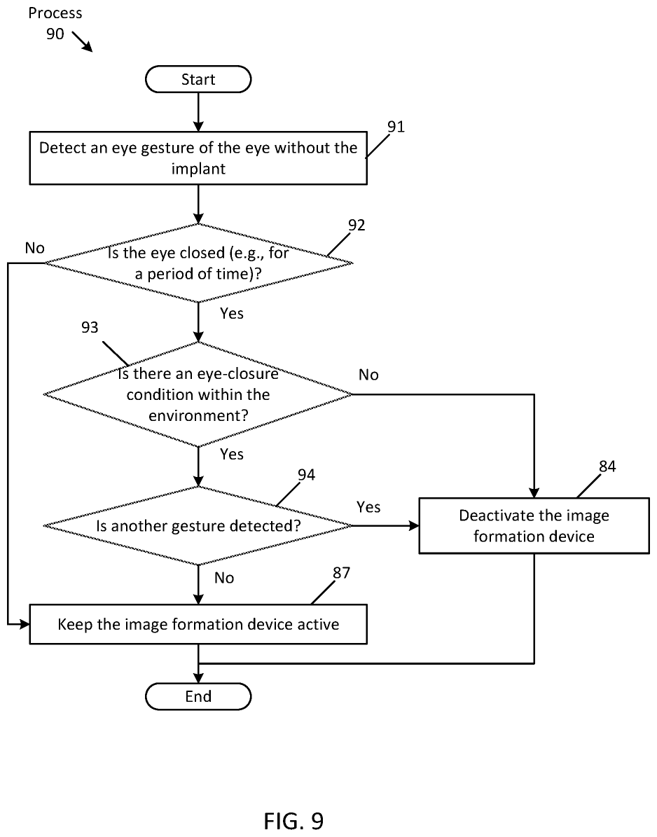

FIGS. 8 and 9 are flowcharts of embodiments of processes 80 and 90, respectively to determine whether to deactivate the image formation device 33 of the implant 3. In one embodiment, at least some of the operations performed by either (or both) of these processes may be performed at blocks 74 and 75 of process 70 in FIG. 7.

Turning to FIG. 8, the process 80 begins by the controller 21 receiving video data captured by a camera with a FOV that includes one or both eye(s) of the user (at block 81). For example, the camera 6 may include a FOV that is directed away from the user when the headset is worn by the user, and the headset 2 may include another camera with a FOV that is directed towards at least a portion of the user's face, while the headset is worn by the user. The controller 21 detects an eye gesture based on the video data (at block 82). For instance, the controller may perform a (eye) gesture detection algorithm to detect an eye gesture of at least one eye of the user. In one embodiment, an eye gesture may be any type of movement or lack thereof (e.g., for a period of time).

The controller determines if the detected eye gesture is a series of (e.g., one or more) eye movements and/or a gaze (at decision block 83). For instance, the controller may monitor the video data for a period of time to determine whether the user is blinking, where each blink may be an eye gesture in which an eye closes and opens (or vice a versa) over a first period of time. As another example, the controller 21 may determine whether the user is winking, where each wink may be an opening and closing gesture performed over a second period of time that is longer than the first period of time. As another example, the controller may determine whether the eye gesture is a gaze in which the user is looking towards a particular direction for a period of time (e.g., without closing the eye). In particular, the controller 21 may perform an eye tracking algorithm to measure eye positions and/or eye movement of at least one eye in a digital image to determine a direction (or point) of gaze with respect to a reference point. In one embodiment, the eye tracking algorithm may determine a direction of gaze based on optical tracking of corneal reflections. For example, (e.g., visible, near-infrared, infrared, etc.) light may be directed towards one or both of the eyes of the user, causing reflections in the cornea. A camera of the headset may capture the reflections, from which a direction of gaze may be determined with respect to the headset (e.g., a position of the camera that is capturing the images). In another embodiment, the controller may determine the direction of the gaze by keeping track of movements of the (e.g., pupils of the) eyes. In another embodiment, the controller 21 may determine the gaze based on performing a head tracking algorithm. In which case, the controller may receive motion data from the accelerometer to determine the position and/or orientation of the user's head. Based on the orientation, the controller may determine a direction towards which the user may be looking. In another embodiment, the eye tracking algorithm and/or head tracking algorithm may use any method to determine the direction of gaze of a person.

In one embodiment, upon detecting that the eye gesture is a series of eye movements (e.g., a series of blinks) or a gaze, the controller may determine whether the detected gesture is associated with a user desire to deactivate the image formation device. For instance, the controller may use the detected eye gesture to perform a table look into a data structure that associates eye gestures with an indication that the image device is to be deactivated (and/or activated). Upon detecting the series of movements and/or gaze, the controller 21 deactivates the image formation device (at block 84). For instance, as described herein, the headset 2 may transmit a control signal to the implant 3 to deactivate the image formation device 33.

If, however, the eye gesture is not a series of movements and/or a gaze, the controller 21 determines whether both eyes are closed (at decision block 85). In particular, the controller may determine whether the eye gesture is both eyes closing and/or remaining closed for a period of time, which may be the case when the user is attempting to go to sleep. If so, the image formation device may be deactivated.

If, however, both eyes (at least one eye) is open, the controller 21 determines whether another gesture is detected (at decision block 86). In particular, the controller may determine whether the user is performing a gesture that indicates a desire to deactivate the image formation device, even though one or both of the eyes are open. As an example, the user may be watching a motion picture, and wish to not view a scene of the motion picture, but would prefer not to close their eyes. As a result, the user may perform another gesture in order to deactivate the implant, while keeping one or both eyes open. In which case, the controller 21 may determine whether the system is receiving user input via one or more input devices. If so, the controller may deactivate the image formation device 33, returning to block 84. If, however, the other gesture is not detected, the controller may keep the image formation device active (at block 87). In particular, the controller 21 may continue to transmit the first video stream captured by the camera 6 to the implant. In one embodiment, the controller 21 may transmit a control signal to the implant, indicating that the implant is to continue display the first video stream.

Some embodiments may perform variations to the process 80 described herein. For example, the specific operations of the process may not be performed in the exact order shown and described. The specific operations may not be performed in one continuous series of operations and different specific operations may be performed in different embodiments. For example, upon determining that both eyes are closed, the controller may determine whether another gestures is detected that indicates that the user wishes to keep the image formation device active. In particular, the controller may determine that the image formation device is to remain active while both eyes are closed, responsive to detecting another gesture, such as detecting user input. This may allow the device to present a scene to the user, while the user's eyes are closed.

In one embodiment, the controller may deactivate the image formation device based a detection of a series of blinks or winks. For instance, this determination may be made by comparing a detection of a series of blinks with a predefined series of blinks, which may be user-defined or may be learned over a period of time. In another embodiment, the determination may be based on whether the series of blinks are known to occur before a person who has normal vision closes their eyes for a period of time.

Turning now to FIG. 9, this figure is a flowchart of the process 90 to determine whether to deactivate the image formation device based on eye gestures performed by the eye of the user without the implant. In particular, these operations may be performed by the intraocular system 20 of a user who is unilaterally blind, where the implant may be inside the eye that is blind. The process 90 begins by the controller 21 detecting an eye gesture of the eye of the user without the implant (at block 91). In which case, video data captured by a camera that is directed towards the user may include a scene of the user's face and including both eyes of the user, where one of the eyes doesn't include the implant. The controller 21 determines if the eye (without the implant) is closed (e.g., for at least period of time) (at decision block 92). If not, the controller may keep the image formation deice active (at block 87). In one embodiment, the controller may detect that eye gesture as the user's eye blinking or winking, where the eye is closed for less than the period of time.

If, however, the eye is closed for at least the period of time, the controller 21 determines if there is an eye-closure condition within the environment (at decision block 93). In one embodiment, an “eye-closure condition” may be a state that a person would close their eyes in response to an external (e.g., environmental) event (e.g., other than a natural blink). Examples of an eye-closure condition may include an environment condition that causes low visibility (e.g., high winds with particulates in the air), a lighting condition with a brightness that exceeds a brightness threshold (e.g., a bright light shining in the user's eyes), etc. In one embodiment, the controller 21 may detect an eye-closure condition within the environment based on sensor data captured by one or more sensors 9. For example, the controller 21 may monitor the video stream captured by the camera 6 to detect environmental conditions (e.g., using an object recognition algorithm). In another embodiment, the controller 21 may detect the condition based on the user's speech, such as detecting using a microphone of the system the user saying, “It is too bright out here!”. If not, meaning that that the user's eyes are closed for a long period of time without any external reason (e.g., the user may be trying to sleep), the controller may deactivate the image formation device (at block 84).

If, however, there is an eye-closure condition, the controller 21 determines whether another gesture (e.g., by the user) is detected (at decision block 94). In particular, the controller determines whether the user has performed another gesture, such as provided user input via one or more input devices. The controller may determine that the image formation device is to remain active, responsive to determining that the user has not performed another gesture, detecting the eye-closure condition, and/or detecting that the user's eye is closed. Thus, the controller 21 may keep the image formation device active (at block 87). In particular, the controller may continue to transmit the video stream to the implant for presentation by the image formation device. Thus, the intraocular system may allow a user to continue viewing the scene in front of the user, even in situations in which a person with normal vision would not be able to see. Responsive, however, to determining that the user has performed another gesture, the detection of the eye-closure condition, and/or that the user's eye without the implant is closed, the controller 21 may deactivate the image formation device (at block 84).

As described herein, the intraocular system determines whether the image formation device is to be deactivated based on one or more detected gestures, where the image device may be deactivated by ceasing to display video data onto the user's retina. In another embodiment, rather than deactivating the image device completely, the system may display (e.g., on the micro-display of the image formation device) a dark background with no light stimulus based on the detected gesture. In particular, the system may display (present) a static (or at least partially) static image (for at least a period of time). For example, upon determining that both of the user's eyes are closed, indicating that the user is trying to sleep, the intraocular system may display an image of a stary night. In one embodiment, the controller 21 may transmit one or more images to the implant upon determining that the image device is to be deactivated, and may cause the implant to display the one or more images.

As described thus far, at least some of the operations described herein may be used to either keep the image formation device active or to deactivate the image formation device. In another embodiment, at least some of the operations may be performed to activate the image formation device from a deactivated state. For example, upon detecting an eye gesture of one or both of the user's eyes opening (e.g., and remaining open for a period of time), the controller may transmit a control signal and/or a captured video stream to the implant to cause the implant to present the video stream onto the user's retina.

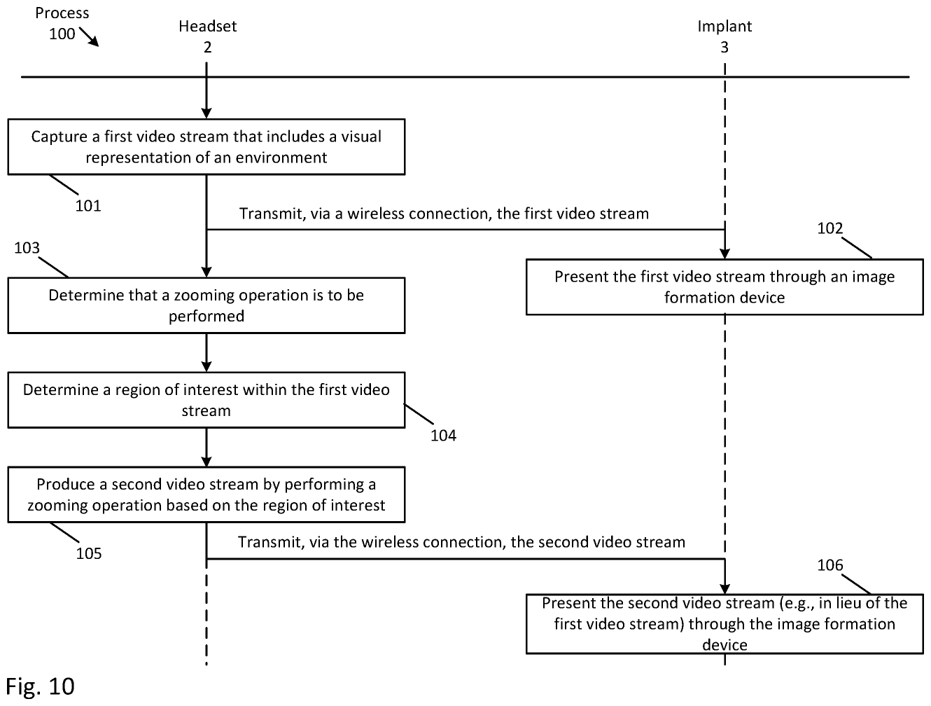

FIG. 10 is a signal diagram of one embodiment of a process 100 by the headset 2 and the implant 3 of the example intraocular system 20 to perform a zooming (or zoom) operation. The process 100 begins by the controller 21 of the headset 2 capturing a first video stream that includes a visual representation of an environment in which the user of the system is located (at block 101). In particular, the controller 21 may receive the first video stream, which is captured by the camera 6 of the headset, while the headset is being worn by the user. The controller 21 transmits, via a wireless connection (e.g., as the data signal 48 shown in FIG. 4), the first video stream to the implant (e.g., via the data antenna 46). The controller 41 of the implant 3 receives the first video stream, via the data antenna 34. The controller 41 presents the first video stream through the image formation device 33 (at block 102). Thus, the implant 3 may be configured to cause the image device 33 to present the first video stream (e.g., displaying the stream on a micro-display, as described herein).

Returning to the headset 2, the controller 21 determines that a zooming operation is to be performed (at block 103). In one embodiment, this determination may be based on a detected gesture that is performed by the user, such as receiving user input through one or more input devices. In one embodiment, the gesture may be an affirmative (voluntary) action (gesture) taken by the user in order to cause the system to perform a zoom operation. For instance, the user may perform a hand gesture that may be detected by the controller 21, using image data captured by one or more cameras, and may determine that a zooming (e.g., zoom-in) operation is to be performed based on the hand gesture (e.g., by comparing the detected hand gesture to a data base (e.g., stored in memory of the headset) of hand gestures that are associated with zooming operations). In another embodiment, the determination may be based on an involuntary action (or gesture) performed by the user. More about this determination is described herein.

The controller 21 determines a region of interest (RoI) within the first video stream (at block 104). In particular, the controller may determine an area (e.g., a 2D area of pixel values) within one or more video frames of the first video stream into which the controller 21 is to zoom (e.g., enlarge). In one embodiment, the RoI may be based on an amount of zoom that is to be performed. For example, the determination that a zooming operation is to be performed may be based on user input, indicating that an amount of magnification (e.g., 2x, 3x, etc.)

is to be performed. In which case, the RoI may be based on the amount of magnification (e.g., where the RoI is centered at a visual center of one or more video frames of the first video stream). In another embodiment, the RoI may be a predefined area. In another embodiment, the RoI may be based on one or more gestures. For instance, the RoI may be determined based on user input, or based on eye gestures (e.g., centered at a location at which a user is looking at based on the user's gaze). More about determining the RoI is described herein.

The controller produces a second video stream by performing a zooming operation based on the region of interest (at block 105). Specifically, the controller may zoom into the RoI. In one embodiment, the controller may perform an optical zoom operation and/or a digital zoom operation. For example, the controller may cause optics (if any) of the headset to adjust one or more lens (e.g., changing a focal length of one or more lenses) to zoom magnify the image. In addition or in lieu of the optical zoom, the controller 21 may perform a digital zoom operation by performing one or more video processing operations upon one or more video frames of the first video stream. In particular, the controller may crop a portion of a video frame based on the determined RoI, and enlarge the RoI based on the resolution and/or aspect ratio of the original video frame (based on the resolution/aspect ratio of the image formation device). In one embodiment, the controller 21 may perform any digital zoom function to enlarge at least a portion of the first video stream. With the produced second video stream, the controller 21 transmits, via the wireless connection, the second video stream to the implant 3. The controller 41 of the implant receives the second video stream, and presents the second video stream (e.g., in lieu of the first video stream) through the image formation device (at block 106).

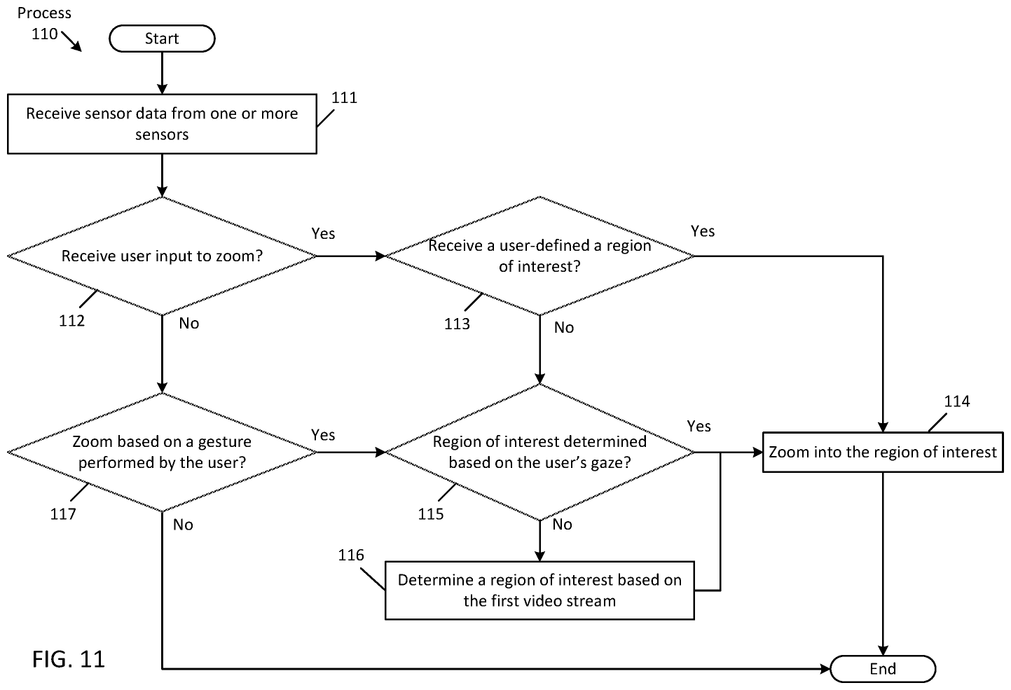

FIG. 11 is a flowchart of one embodiment of the process 110 to determine whether to zoom into the video stream captured by the camera. In particular, this process 110 describes operations in which one or more zooming operations may be performed automatically by the intraocular system 20 (e.g., without user intervention and/or based on involuntary actions/gestures performed by the user). The process 110 begins by the controller 21 receiving sensor data from one or more sensors, such as the sensors 9 shown in FIG. 4 (at block 111). In one embodiment, the sensor data may include video data captured by one or more cameras with different FOVs, such as one camera having a FOV that is directed towards one or both of the user's eyes, while the headset 2 is being worn by the user. In another embodiment, the senor data may include motion data produced by the accelerometer 7 of the headset.

The controller 21 determines whether user input to zoom has been received (at decision block 112). For example, the input device 11 of the headset 2 may include one or more interfaces (e.g., a push-button or a capacitive touch control, which may be a button or a slider) that allows the user to zoom in (and/or out) of the video that is being displayed by the implant 3 (e.g., while user input is being received through the interfaces). In one embodiment, the input device 11 of the headset 2 may provide the zooming operation, while user input is being received, such that when user input is no longer being received, the controller may zoom back out of the video.

If so, the controller 21 determines whether a user-defined region of interest (Rol) has been received (at decision block 113). In particular, the controller 21 determines whether user input has been received that indicates a RoI in which the user wishes the intraocular system to zoom. In one embodiment, the user-defined RoI may be based on user input that indicates an amount of magnification (zoom) that may be requested based on the user input. An example may include a voice command such as “System, please magnify by 50%”. In some embodiments, when the user input is a selection of a push-button on the headset 2, the longer the user depresses the push-button, the more the intraocular system may zoom into the first video stream. As a result, the size of the region of interest may be proportional to the amount of zoom that is requested by the user (e.g., the size of the RoI decreases as the amount of desired zoom increases), where the RoI may be centered (e.g., with respect to one or more video frames) to the first video stream.

In another embodiment, the RoI may be defined based on a user command. In particular, the user may provide instructions to the controller 21 to zoom into a particular portion of the first video stream. For example, the user input may include a voice command of the user indicating the RoI as a location and/or object, such as “Zoom into the red sign.”. In which case, the controller 21 may be configured to perform an object recognition function to identify a red sign within the scene of the first video stream. Once identified, the controller 21 may be configured to define the RoI to include at least a portion of the red sign. If the RoI is user defined, the controller 21 zooms into the user-defined RoI (at block 114). In particular, the controller 21 may perform an optical zoom and/or digital zoom in order to magnify the video stream into the user-defined RoI.

In one embodiment, the controller may be configured to zoom up to a threshold, beyond which the controller may not zoom in order to preserve picture quality. Specifically, the threshold may be based on the resolution of the image formation device 33. For example, if the camera resolution is greater than the image formation resolution (e.g., the camera resolution being 3,000,000 pixels, while the micro-display of the image formation device is 300,000 pixels), the controller may reduce the amount of digital zoom that is available in order to avoid pixel interpolation.

Returning to decision block 113, if, however, a user-defined RoI is not received, the controller 21 determines if the RoI may be determined based on the user's gaze (at decision block 115). In particular, the controller 21 may receive video data from a camera with a FOV that includes one or both of the user's eyes, and may perform an eye tracking algorithm to determine a direction (a location and/or an object within the environment) at which the user is looking. Upon determining the user's gaze, the controller may determine the RoI. Continuing with a pervious example, the controller may determine that the user is looking at a red sign within the environment based on the direction of gaze with respect to the video data that is being presented to the user. The controller 21 may then zoom into the RoI, at bock 114.

In one embodiment, the controller may determine the gaze of at least one eye to determine the RoI. In another embodiment, the controller may determine the gaze of both eyes (e.g., simultaneously), which may provide a better indication of a RoI. For example, by determining the gaze of both eyes, the controller may be configured to converge to a more specific direction (or location) at which the user is looking, and thus provide a zoom to view a smaller angular field of view.

In another embodiment, the RoI may be determined based on other types of gestures. For example, the RoI may be determined based on a physical gesture, such as a hand gesture (e.g., where the user is pointing towards a location or object), a head gesture, etc.

If, however, the region of interest may not be determined based on a user's gaze, the controller 21 may determine the region of interest based on the first video stream that is being presented to the user (at block 116). For example, the user's gaze may not be determined when the user's eyes are closed, or may not be fixated towards a particular direction (e.g., for a period of time). In which case, the controller 21 may be configured to determine the region of interest based on the video that is being presented to the user. For instance, the controller may define the RoI upon an identified object within the scene that is being presented to the user. In particular, the controller may determine that an object is of interest to the user based on being presented to the user for a period of time. As another example, the controller may define the RoI upon a moving object. Upon determining the RoI, the controller may zoom into the RoI, at block 114.

Returning to decision block 112, if user input is not received to zoom, the controller determines whether a zooming operation may be performed based on a gesture performed by the user (at decision block 117). In which case, the gesture may be an involuntary gesture or a voluntary gesture. For example, the controller 21 may detect an eye gesture, such as a series (or a pattern) of one and/or more blinks or eye movements, and may determine whether to zoom based on the detected eye gesture (e.g., performing a table lookup into a data structure that associates zooming instructions with eye gestures). In one embodiment, the eye gesture may include a pattern of one or more winks, which may include eye movements of one or both of the user's eyes that are performed individually with respect to each other. In another embodiment, the determination may be based on multiple gestures. For instance, the controller may determine whether to zoom based on detecting a user's gaze and detecting one or more eye movements. In another embodiment, the detected gesture may be any type of physical gesture, such as a hand movement, a head movement, or a facial expression. Upon detecting the gesture, the controller may proceed to determining the region of interest at decision block 115.

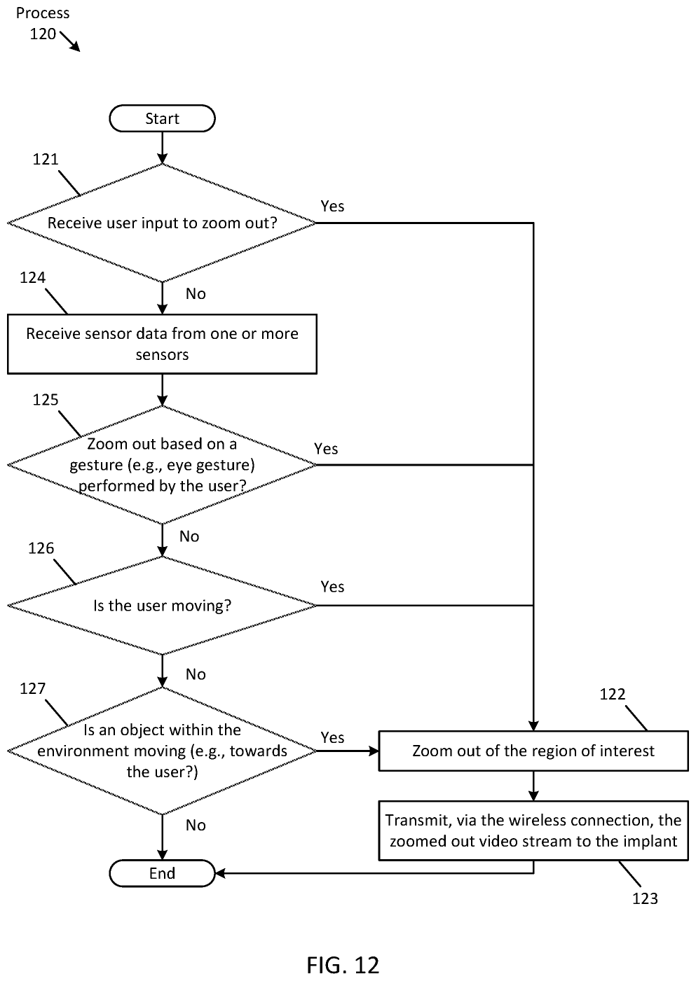

FIG. 12 is a flowchart of one embodiment of a process 120 to determine whether to perform a zoom-out operation. In particular, the process 120 may be performed by the controller 21 to determine whether a RoI into which the controller had previously zoomed into (e.g., based on the controller performing the operations of process 110 of FIG. 11) is to be zoomed out. Thus, this process may be performed such that the RoI into which the captured video stream was zoomed into, thereby producing a zoomed-in video stream that is being displayed to the user, may be zoomed out of, such that the intraocular system may present a larger field of view (e.g., the original field of view) of the scene captured by one or more cameras. In one embodiment, at least some of the operations of process 120 may be performed (e.g., after a period of time) after the process 110 of FIG. 11 is performed to zoom into a RoI and/or while the user is viewing the zoomed-in video stream.

The process 120 begins by the controller 21 determining whether user input has been received to performing a zooming out operation (at decision block 121). For example, the controller may be configured to determine whether the RoI is to be zoomed out (e.g., to present a non-zoomed view of the scene captured by camera 6) based on whether user input is received. For example, the input device 11 may receive user input by the user depressing one or more physical buttons (or releasing a depressed button), for example. As another example, the controller 21 may receive a voice command from the user, such as “Zoom out.” In one embodiment, the user input may indicate a magnification to which the system is to zoom out.

If user input is received, the controller 21 zooms out of the region of interest (at block 122). Specifically, the controller may produce another video stream by zooming out of the RoI. For example, the controller may perform a zoom-out operation upon the zoomed-in video stream. In one embodiment, the produced zoomed-out video stream may be the original video stream (e.g., the first video stream) captured by the camera 6. In another embodiment, the produced video stream may be a magnified video stream that has a magnification between the originally captured video stream by the camera 6 and the zoomed-in video stream that the user is viewing before the RoI is zoomed out. In one embodiment, the controller may determine another (zoomed-out) RoI of the original video stream, which may be larger than the currently zoomed-in RoI, to which the video stream is to be zoomed out. In one embodiment, the zoomed-out RoI may correspond to the resolution of the first video stream that is displayed by the image formation device without any zooming operation performed by the surgical system. In another embodiment, the zoomed-out RoI may be a predefined magnification of the original video stream.

The controller 21 transmits, via the wireless connection, the zoomed-out video stream to the implant in lieu of the zoomed-in video stream for display on the image formation device (at block 123). In one embodiment, the controller may apply the zoom-out operation over a period of time, such that the user may view the operation (e.g., as an animation)

If, however, user input is not received, the controller receives sensor data from one or more sensors, such as sensors 9 of the headset (at block 124). The controller determines whether to zoom out based on a gesture, such as an eye gesture, performed by the user based on the sensor data (at decision block 125). For example, the controller may determine whether to zoom out based on a series (or pattern) of detected blinks, winks, and/or eye movements by an eye tracking algorithm, as described herein. If so, the controller proceeds to zoom out of the region of interest at block 122. In one embodiment, the controller may determine a new RoI to which the controller is to zoom out based on the sensor data. For instance, the RoI may be determined based on the detected gesture. As an example, when the gesture is a hand movement that mimics adjusting a physical dial, knob, or slider, the controller may determine the amount at which the video is to be zoomed out based on the hand movement (e.g., based on the rotation or translation of the user's hand).

If, however, a gesture is not detected, the controller determines whether the user is moving (at decision block 126). In particular, the controller may monitor sensor data to determine whether the user is moving from a (e.g., stationary) position at which the user's vision was magnified. Specifically, the controller may determine whether the RoI is to be zoom out by determining whether the user is in motion. Thus, the system may provide a far-end view when the user wishes to view objects or locations far away, while ensuring that the system provides a near-end field of view of the scene of the environment as the user moves (walks) about the environment (e.g., in order to avoid collisions with near-by objects). If the user is moving, the controller may be configured to zoom out of the RoI, at block 122.

If, however, the user is not moving, the controller 21 determines if an object within the environment is moving, for example towards the user (at decision block 127). Specifically, the controller 21 may be configured to deactivate zoom (e.g., zooming out the user's view to the original magnification of the video stream captured by the camera 6) if an object is detected nearby the user. For example, the controller may be configured to receive proximity data from the proximity sensor 8, and may determine that the region of interest is to be zoomed by based upon determining that an object is within a threshold distance of the user based on the proximity data. Continuing with the previous example, the system may zoom out while a person is in a stationary position looking at a far-away sign, responsive to detecting an object, such as a person or an animal that is approaching the user. This may allow the user to view the approaching object and enough time to react (e.g., to move out of the way). In another embodiment, in addition to detecting that an object is moving within the environment, the controller may deactivate the zoom if the object is moving towards the user at a certain speed. For instance, the controller may monitor video data captured by one or more motion sensor cameras to detect a rate of speed at which an object (e.g., a bicycle) is approaching the user, and may determine that the video is to be zoomed out if the rate of speed is greater than a threshold value. If an object is moving towards the user (and/or at a particular rate of speed), the controller may perform a zoom-out operation, as described herein.

In one embodiment, the controller 21 may determine that the RoI is to be zoomed out based on one or more conditions described herein. For instance, the controller may deactivate the zoom in response to detecting that the user is moving and that an object is moving towards the user.

As described thus far, the intraocular system may zoom out of a RoI in which the system is currently zoomed into. In another embodiment, at least some of the operations of process 110 may be performed to zoom out of the scene within the first video stream captured by the camera 6 upon which no magnification is currently being performed. In which case, the RoI may be a predefined region.

The intraocular system may determine whether to deactivate the image formation device and/or perform a zoom-in/zoom-out operations based on sensor data and/or user input. In another embodiment, the system may determine whether to perform other operations, such as performing video processing operations upon the video stream captured by the camera 6 and displayed by the micro-display of the implant. For example, the sensors 9 may include an ambient light sensor that may be used by the system to determine if the visualization should switch from a standard visual imaging (e.g., in bright light and in color images) to motion detection or infrared images (e.g., in low light conditions) based on the sensor data and/or user input. As another example, the switch may be based on a detected gesture. For example, upon detecting a hand gesture, such as forming a visor over the user's brow (indicating that the user is in a bright environment), the controller 21 may perform a brightness adjustment to the video stream. Upon detecting another gesture, such as the user moving the hands away from the brow of the user's face, the controller may be configured to remove the brightness adjustment, indicating that the user has moved away from the bright environment.

FIG. 13 is a flowchart of one embodiment of a process performed by the implant 3 to determine process a received video stream based on sensor data. In particular, the controller 41 of the implant may perform at least some operations described herein. The process 130 begins by the controller 41 receiving, via a wireless connection, a video stream (at block 131). In particular, the implant 3 may receive the video stream from the headset 2, where the stream is captured by one or more cameras (e.g., camera 6) of the headset that is being worn by the user. The controller 41 (optionally) causes the image formation device 33 of the implant 3 to present the received video stream (at block 132). Specifically, the controller 41 may display the video stream on a micro-display of the image formation device, such that the video is formed onto at least a portion of the user's retina.

The controller 41 receives sensor data from an intraocular sensor (e.g., sensor 27) that is implanted into the eye of the user (at block 133). In particular, the controller 41 may receive an electrical signal from the sensor 27, which may be implanted within a portion (e.g., a muscle) of the user. For example, responsive to the user performing an eye gesture, such as a wink, the sensor may produce the signal and transmit the signal to the controller 41. The controller 41 determines an eye gesture performed by the user based on the sensor data (at block 134). Continuing with the previous example, responsive to receiving the signal, the controller may determine the eye gesture. As an example, the controller may determine that the user's eye is closed (e.g., for a period of time) based on the electrical signal, may determine the direction of gaze based on the electrical signal, and/or may determine one or more eye movements based on the electrical signal.

The controller 41 determines a video processing operation based on the detected eye gesture (at block 135). For instance, the controller may determine that the user desires the intraocular implant to mimic a low-light environment by detecting that the user's eyes have closed (e.g., for a period of time). In one embodiment, a low-light environment may include the image formation device being deactivated (turned off), or may include the device displaying the video stream at a lower brightness setting, or may include the image device displaying an image for a period of time, as described herein. In one embodiment, the image device may display a low-light environment may displaying an image (e.g., a starry night scene), while the image formation device has a brightness below a threshold.

In another embodiment, the controller may determine that another video processing operation may be performed based on the detected eye gesture. For instance, the controller may determine that a zoom operation is to be performed responsive to the eye gesture detection. In which case, the controller may be configured to determine a region of interest in which to zoom based on the sensor data. For example, the controller may determine a direction of gaze based on the sensor data, where the region of interest includes a portion of the first video stream that is in the direction of the gaze.

The controller 41 produces a processed video stream by performing the video processing operation upon the video stream (at block 136). For example, the controller may produce the processed stream by zooming into (e.g., a determined region of interest) the first video stream based on the sensor data (e.g., based on the direction of gaze determined according to a detected eye gesture). The controller 41 causes the image formation device 33 (e.g., micro-display) to present the processed video stream (e.g., in lieu of the received video stream) (at block 137). As another example, upon determining that the eye gesture indicates a user-desire to view a low-light environment, the controller 41 may cause the image formation device to display one or more images (e.g., for a period of time).

As described herein, the controller 41 may process the received video stream based on sensor data from the intraocular sensor 27. In another embodiment, the controller may receive sensor data from one or more other sensors. For example, the implant 3 may receive sensor data from one or more of the sensors 9 of the headset 2. In yet another embodiment, rather than (or in addition to) the controller determining 41 that the video is to be processed, the controller 21 of the headset may make this determination. In which case, the controller 41 may be configured to receive a control signal from the headset 2 that includes instructions for the implant to perform the one or more video processing operations, such as a digital zoom operation.

Some embodiments may perform variations to the process 60 described herein. For example, the specific operations of the process may not be performed in the exact order shown and described. The specific operations may not be performed in one continuous series of operations and different specific operations may be performed in different embodiments. For example, upon determining that the image formation device is to be deactivated, based on a detected eye gesture at block 134, the controller 41 may cease displaying the received video stream on the micro-display. This may be the case when it is determined that the user desires for the implant to mimic a low-light environment.

In one embodiment, processing the received video based on sensor data from one or more intraocular sensors has several advantages. First, due to at least some electronics being at the user-side, the headset may include less electronics (e.g., less sensors), and therefore be less bulky. Second, processing the video based on eye gestures detected by an intraocular sensor may allow the user to enhance the video privately. For instance, rather than receiving user input through the input device 11 of the headset, the system may process the video based on eye gestures at the implant. This may make the processing less obvious to outside observers.

As described herein, the intraocular system may perform video processing operations upon captured video. In one embodiment, the system may process video based on a user's visual capabilities. For example, when a user is unable to discern one or more colors, due to being at least partially color blind, the system may perform color processing operations to compensate for the one or more colors. In particular, the controller may present the one or more colors as other colors that the user may be able to discern. For example, when a user has red-green color blindness, the controller may adjust one or more color characteristics (e.g., the hue, brightness, and/or chroma).

An embodiment of the present disclosure includes a headset that is arranged to be worn by a user, wherein the headset includes: a camera to capture video of an environment in which the headset is located, a controller configured to produce processed video according to one or more video processing operations, a wireless data transmitter to wirelessly transmit the processed video, while the headset is worn by the user; and an implant that is inside an eye of the user, the implant including: a wireless data receiver to wirelessly receive the processed video, and an image formation device to present the processed video towards a retina of the eye.

An embodiment of the present disclosure is a method performed by an intraocular implant that comprises a micro-display, the method including: receiving, via a wireless connection and while the intraocular implant is inside an eye of a user, a video stream that is captured by a camera of a headset that is being worn by the user; displaying the video stream on the micro-display; determining that the user desires the intraocular implant to mimic a low-light environment; and responsive to determining that the user desires that the intraocular implant mimic the low-light environment, ceasing to display the video stream on the micro-display.

In one embodiment, determining that the user desires the intraocular implant to mimic the low-light environment includes detecting a gesture performed by the user. In another embodiment, detecting the gesture includes receiving an electrical signal from an intraocular sensor; and determining that the user's eye is closed based on the electrical signal. In another embodiment, the method further includes displaying an image on the micro-display for a period of time responsive to the determining that the user desires that the intraocular implant mimic the low-light environment. In some embodiments, the intraocular implant mimics the low-light environment by the micro-display having a brightness below a threshold while displaying the image.

An embodiment of the present disclosure is a method performed by an intraocular implant that comprises a micro-display, the method includes receiving, via a wireless connection and while the intraocular implant is inside an eye of a user, a first video stream that is captured by a camera of a headset that is being worn by the user; displaying the first video stream on the micro-display; receiving sensor data from an intraocular sensor that is inside the eye of the user; producing a second wireless stream by zooming into the first video stream based on the sensor data; and transitioning from displaying the first video stream to the second video stream on the micro-display.

In one embodiment, the method further includes detecting an eye gesture of the eye based on the sensor data, where the second wireless stream is produced responsive to the detected eye gesture. In another embodiment, producing the second wireless stream by zooming includes determining a region of interest into which to zoom based on the sensor data. In some embodiments, determining the region of interest includes detecting a direction of gaze based on the sensor data, where the region of interest includes a portion of the first video stream that is in the direction of the gaze.

Some embodiments may perform variations to one or more processes described herein. For example, the specific operations of the process may not be performed in the exact order shown and described. The specific operations may not be performed in one continuous series of operations and different specific operations may be performed in different embodiments. In one embodiment, at least some of the operations of one or more processes described herein may be optional. In particular, blocks that have dashed boundaries may be optionally performed when their respective processes are performed. For example, the operations to cause the image formation device to present the video stream at block 132 of process 130 in FIG. 13 may be optionally performed by the implant.

As previously explained, an embodiment of the disclosure may be a non-transitory machine-readable medium (such as microelectronic memory) having stored thereon instructions, which program one or more data processing components (generically referred to here as a “processor”) to perform the (video) signal processing operations, as described herein. In other embodiments, some of these operations might be performed by specific hardware components that contain hardwired logic. Those operations might alternatively be performed by any combination of programmed data processing components and fixed hardwired circuit components.

While certain embodiments have been described and shown in the accompanying drawings, it is to be understood that such embodiments are merely illustrative of and not restrictive on the broad disclosure, and that the disclosure is not limited to the specific constructions and arrangements shown and described, since various other modifications may occur to those of ordinary skill in the art. The description is thus to be regarded as illustrative instead of limiting.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

In some embodiments, this disclosure may include the language, for example, “at least one of [element A] and [element B].” This language may refer to one or more of the elements. For example, “at least one of A and B” may refer to “A,” “B,” or “A and B.” Specifically, “at least one of A and B” may refer to “at least one of A and at least one of B,” or “at least of either A or B.” In some embodiments, this disclosure may include the language, for example, “[element A], [element B], and/or [element C].” This language may refer to either of the elements or any combination thereof. For instance, “A, B, and/or C” may refer to “A,” “B,” “C,” “A and B,” “A and C,” “B and C,” or “A, B, and C.”

What is claimed is:

1. A method performed by a headset that is part of an intraocular system, wherein the intraocular system comprises an implant that includes an image formation device, the method comprising:
capturing, by a camera of the headset that is being worn by a user, a first video stream that includes a visual representation of an environment of the headset;
determining whether the first video stream is to be enhanced;
responsive to determining that the first video stream is to be enhanced, producing a second video stream that includes an enhanced visual representation of the environment; and
transmitting, via a wireless connection, the second video stream to the implant that is inside an eye of the user, wherein the implant is configured to use the image formation device to present the second video stream.

2. The method of claim 1 further comprising receiving, from a proximity sensor of the headset, proximity data that indicates a distance from an object within the environment, wherein determining whether the first video stream is to be enhanced comprises determining that the distance is within a threshold distance.

3. The method of claim 2, wherein the enhanced visual representation of the environment comprises a notification to notify the user of a presence of the object within the environment.

4. The method of claim 1, wherein determining whether the first video stream is to be enhanced comprises detecting, using an object recognition algorithm, an object within the environment, wherein producing the second video stream comprises adding a notification relating to the object to a portion of the first video stream.

5. The method of claim 4, wherein adding the notification comprises highlighting a region surrounding the object within the video stream to emphasis the object.

6. The method of claim 1, wherein the image formation device comprises a micro-display with a plurality of pixel arrays.

7. The method of claim 1, wherein the image formation device comprises a laser projector.

8. A method performed by a headset that is part of an intraocular system, wherein the intraocular system comprises an implant that includes an image formation device, the method comprising:
capturing, by a camera of the headset that is being worn by a user, a video stream that includes a visual representation of an environment in which the headset is located;
transmitting, via a wireless connection, the video stream to the implant that is inside an eye of the user, wherein the video stream is for presentation by the image formation device;
detecting a gesture performed by the user;
determining whether the image formation device is to be deactivated or remain active based on the detected gesture; and
transmitting, via the wireless connection and responsive to determining that the image formation device is to be deactivated, a control signal to the implant to cause the image formation device to deactivate.

9. The method of claim 8, wherein the camera is a first camera that has a first field-of-view (FOV) directed away from the user when the headset is worn by the user, wherein the headset comprises a second camera that has a second FOV directed towards the eye of the user when the headset is worn by the user, wherein detecting the gesture comprises performing a gesture detection algorithm to detect an eye gesture of the eye of the user.

10. The method of claim 9, wherein the image formation device is determined to be deactivated when the eye gesture is a gaze for a period of time.

11. The method of claim 9, wherein the image formation device is determined to be deactivated when the eye gesture is a series of blinks or winks.

12. The method of claim 9, wherein the second FOV is directed towards both eyes of the user, wherein the eye gesture comprises both eyes closing, wherein the image formation device is determined to be deactivated responsive to determining that both eyes are closed and remain closed for a period of time.

13. The method of claim 12, wherein the gestures is a first gesture, wherein the method further comprises detecting a second gesture performed by the user, wherein the image formation device is determined to remain active while both eyes are closed responsive to the detecting of the second gesture.

14. The method of claim 8 wherein the eye is a first eye, and the gesture is a first gesture, wherein the control signal is a first control signal, wherein the gesture comprises the second eye of the user closing, wherein the method further comprises:
detecting an eye-closure condition within the environment;
determining whether the user has performed a second gesture;
responsive to determining that the user has performed the second gesture, the detecting of the eye-closure condition, and that the user's second eye is closed, determining that the image formation device is to be deactivated.

15. The method of claim 14, wherein determining that the image formation device is to be deactivated comprises ceasing to transmit the video stream to the implant, wherein the method further comprises,
determining that the image formation device is to remain active responsive to determining that the user has not performed the second gesture, the detecting of the eye-closure condition, and that the user's second eye is closed; and
responsive to determining that the image formation device is to remain active, continuing to transmit the video stream to the implant for presentation by the image formation device.

16. The method of claim 14, wherein the eye-closure condition comprises at least one of an environmental condition that causes low visibility, and a lighting condition with a brightness that exceeds a threshold.

17. The method of claim 8, wherein the control signal causes the image formation device to be deactivated by instructing the image formation device to cease displaying the video stream.

18. The method of claim 17 further comprises transmitting, via the wireless connection, an image to the implant to cause the image formation device to present the image.

19. The method of claim 8, wherein the operations are performed by the headset.

20. A headset comprising:
a camera;
a processor;
memory having stored therein instructions which when executed by the processor causes the headset to:
capture, by the camera and while the headset is worn by a user, a video stream that includes a visual representation of an environment of the user;
transmit, via a wireless connection, the video stream to the implant that is inside an eye of the user to display the video stream on a micro-display;
detect a gesture performed by the user;
determine that the micro-display is to display a dark background with no light stimulus based on the detected gesture; and
transmit, via the wireless connection, a control signal to the implant to cause the micro-display to display the dark background.
